(19) United States
(12) Patent Application Publication
(10) Pub. No.: US 2023/0255205 A1
(43) Pub. Date: Aug. 17, 2023

(54) HERBICIDAL HETEROCYCLIC DERIVATIVES

(71) Applicant: MoA Technology Limited, Oxfordshire (GB)

(72) Inventors: Ben ALLEN, Oxfordshire (GB); Ian HOULSBY, Oxfordshire (GB)

(73) Assignee: MoA Technology Limited, Oxfordshire (GB)

(21) Appl. No.: 18/014,948

(22) PCT Filed: Jul. 8, 2021
(86) PCT No.: PCT/GB2021/051751

§ 371 Date: Jan. 6, 2023

(30) Foreign Application Priority Data
Jul. 10, 2020 (GB) 2010678.7

(51) Int. Cl. A01N 43/653; C07D 249/14; A01N 43/82; C07D 271/07; A01P 13/00

(52) U.S. Cl. CPC A01N 43/653; C07D 249/14; A01N 43/82; C07D 271/07; A01P 13/00

(57) ABSTRACT

The present invention relates to the use of compounds of Formula (I) as herbicides, wherein Z, W, X¹-X⁴, R₁, F₂, R₃, R₄, m and n are as defined herein. The invention further relates to agrochemical compositions comprising a compound of Formula (I) and to the use of such compositions for controlling weeds at a locus, particularly among useful crops. The invention further relates to select compounds of Formula (I).

### FIELD OF THE INVENTION

The present invention concerns the use of heterocyclic derivatives as herbicides and compounds of certain novel heterocyclic derivatives. It further concerns agrochemical compositions which may be made using the herbicidal heterocyclic derivatives and a method of controlling weeds at a locus.

### BACKGROUND OF THE INVENTION

The presence of undesired plants, for example weeds, increases demand on resources and effectively reduces the share of resources available to more useful plants, such as crops. This in turn reduces the yields of such crops affected by nearby weed growth. There exists a wide variety of plants commonly regarded as weeds in the context of crop growth, including broadleaf plants and grasses.

In addition to direct competition for resources, weeds are frequently allelopathic, i.e. they produce one or more biochemicals (often as secondary metabolites) which are capable of influencing the germination, growth, survival and reproduction of other organisms nearby. Such organisms can include other plant species or can include animal species. The process of allelopathy is a key element in the distribution of species and competition between them, and is also considered to be a significant weapon in the arsenal of many invasive species. Allelopathic weeds may be capable of inhibiting the growth of crop plants to a greater degree than by resource competition alone.

Agrochemicals, or agricultural chemicals, are those chemicals used for agricultural purposes. They are classified based on the role for which they are being used, e.g. pesticides for the controlling of pests, fungicides for the controlling of fungal growth, fertilisers for enhancing the nutrient content of the soil in which crops are grown, or herbicides, which are used to destroy unwanted vegetation.

Herbicides in particular may be selective or non-selective. The former are herbicides designed for use around desired plants/crops and seek to control weeds without damaging the desired plant/crop itself. The latter are herbicides which do not discriminate on variety of plant, but instead destroy all vegetation. Non-selective herbicides are therefore typically not used on crop fields during the growth of crops.

Herbicides may be applied by a variety of routes and may have a variety of mechanisms of action. They can be applied to the soil, so as to be absorbed by the roots/shoots of emerging weed seedlings, or they can be applied to the leaves of existing plants. The choice of route can also dictate whether a herbicide is a pre-emergence herbicide (i.e. applied before the weed seedlings emerge at the surface) or a post-emergence herbicide (one which is applied after the weed seedlings have emerged through the soil surface). Each type of herbicide has particular considerations with respect to the method of application and how to achieve persistence in the soil.

The use of herbicides must be carefully managed. In general, herbicides are expensive substances and thus an economic motive exists for minimising their use. In addition, herbicide use can have undesirable environmental impact, for example in the contamination of groundwater, animal and human health concerns and in the development of herbicide-resistant weeds. There is therefore an incentive to minimise the quantities of herbicides used in any one area needing weed control. This is not always easy, however, as the development of resistance to existing herbicides requires the use of ever larger quantities of herbicides.

The yields of crop plants can be significantly reduced by weed infestations. For example, redroot pigweed or Amaranthus retroflexus, is an aggressive and highly competitive weed in the growth of many crops. Its unchecked growth induces significant losses in the yields of soybeans, cotton, maize, sugar beet, sorghum among many others (Weaver et al., “The biology of Canadian weeds. 44. Amaranthus retroflexus L., A. powellii S. Wats. and A. hybridus L.”, Can. J. Plant. Sci., 1980, 60, 4, 1215-1234). The damage caused by A. retroflexus is not limited by geography either, indeed the weed is present globally. A. retroflexus has been reported to exhibit allelopathic effects on other weeds and crop plants, further reducing crop yields. It has also been implicated in harm to livestock, for example by facilitating the accumulation of harmful substances (e.g. nitrates and oxalates) in leaves and stems. In addition, A. retroflexus is known to be an additional vector for a range of crop pests and diseases, including parasitic weeds in tomato plants (Orobanche ramosa), aphids in orchards (Myzus persicae) and a cucumber mosaic virus in peppers (Weaver et al.). Many weeds, including A. retroflexus have developed resistance to existing herbicides (Francischini, A., et al. “Multiple- and Cross-Resistance of Amaranthus retroflexus to Acetolactate Synthase (ALS) and Photosystem II (PSII) Inhibiting Herbicides in Preemergence.” Planta Daninha 37 (2019).) Similar problems and issues are encountered with many other weed species.

Accordingly, there is a strong incentive to develop new herbicides, to widen the range of available herbicides and to produce herbicides with superior properties, such as superior herbicidal performance or lower environmental impact. The compounds and compositions of the present invention represent a significant step forward in meeting these goals.

DE3101889 discloses heterocyclic derivatives for use as herbicides, wherein the heterocyclic components are chosen from pyridine, thiazole, thiophene, thiadiazole or triazine residue. It does not disclose the use of compounds containing triazoles, tetrazoles or oxadiazoles. The disclosed 5-membered heterocycles contain sulphur.

Further, it is well known that lignin, a phenolic polymer deposited in a plant's secondary-thickened walls, provides a physical barrier against insects and pathogens. Van de Wouwer et al. established a chemical genetic approach to find candidate inhibitors of lignification in Arabidopsis thaliana, namely heterocyclic compounds. In particular, substituted benzene with a further substituted 1,3 oxazole attached, was found effective at inhibiting lignification in the hypocotyl and the root of Arabidopsis.

CN 103524441 discloses a triazole-containing compound and its preparation via a catalytic electrophilic substitution reaction, whereby the application of said compound is as a herbicide. The active ingredient for herbicidal activity is an amino substituted triazole

U.S. Pat. No. 4,671,818 teaches the compositions of furazans substituted with an alkyl group and a further substituted amide group suitable for herbicidal applications, by applying an optimal amount of 0.1 kg/hectare up to 5 kg/hectare of the compound after the emergence of unwanted weeds. Further disclosed is the preparation of such compounds using the furazan active ingredient and an adjuvant.

JP H0432070 and JP H0580469 disclose novel substituted pyrazole compounds with a pyridine ring attached, capable of selective and highly effective herbicidal activity, as well as exhibiting antibacterial activity. The disclosed compounds have shown efficacy at killing weeds in paddy fields, namely Scirpus hotarui and Sagittaria pygmaea, without impacting the paddy fields themselves.

### SUMMARY OF THE INVENTION

The present invention relates to herbicidally active heterocyclic derivatives. The invention further extends to herbicidal compositions comprising such derivatives, as well as the use of such compounds and composition for controlling undesirable plant growth, such as weeds, and the method involved in such use.

The present invention provides in a first aspect a use of a compound as defined in claim 1 of general Formula (I) or an agriculturally acceptable salt thereof as an agrochemical, preferably a herbicide:

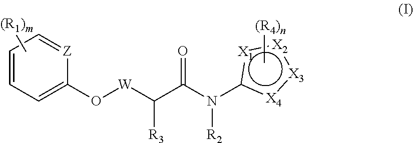

In a second aspect the invention provides an agricultural composition, preferably a herbicidal composition, comprising a compound according to the first aspect of the invention and an agriculturally acceptable formulation adjuvant.

In a third aspect the invention provides a method of controlling weeds at a locus comprising application to the locus of a weed controlling amount of a composition according to the second aspect of the invention.

In a fourth aspect the invention provides a compound or an agriculturally acceptable salt thereof.

### DETAILED DESCRIPTION OF THE INVENTION

According to the present invention there is provided the use as an agrochemical, preferably a herbicide, of a compound of general Formula (I) or an agriculturally acceptable salt thereof:

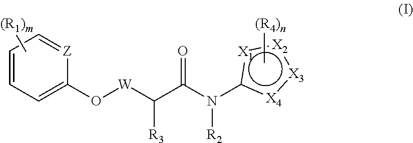

wherein
each R₁ is independently selected from CN, nitro, halogen, OR₆, SR₆, NR₆R₇, NR₆OR₇, NR₆NR₇R₈, ONR₆R₇, ON(═CR₆), R₂₀, OR₂₀, SR₂₀, NR₆R₂₀;
C₁₋₆ alkyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, C₂-C₆ alkenyl and C₂₋₆ alkynyl, any of which may be optionally substituted;
wherein R₁ may independently or together with a further R₁ form a group selected from C₃-C₁₀ cycloalkyl, C₃₋₁₀ heterocycloalkyl, C₃₋₁₀ cycloalkenyl, C₃₋₁₀ heterocycloalkenyl, C₆-C₁₀ aryl or C₅-C₁ heteroaryl which may be optionally substituted;
R₂ is selected from H or C₁-C₄ alkyl;
R₃ is selected from H and optionally substituted C₁-C₄ alkyl,
W is selected from —(C(R₃)₂)ₚ— wherein p is 0, 1, 2 or 3;
Z is selected from CH, CR₁ and N;
X¹-X⁴ are independently selected from CH, CR₄, N, NH, NR₄ and O;
wherein at least three of X¹-X⁴ are N, NH, NR₄ or O;
such that the ring containing X¹-X⁴ is aromatic;
each R₄ is independently selected from the same groups as listed for R₁,
wherein R₄ may independently or together with a further R₄ form a group selected from C₃-C₁₀ cycloalkyl, C₃₋₁₀ heterocycloalkyl, C₃₋₁₀ cycloalkenyl, C₃₋₁₀ heterocycloalkenyl, C₆-C₁₀ aryl or C₅-C₁₀ heteroaryl which may be optionally substituted;
R₆, R₇ and R₆ are independently selected from the group consisting of hydrogen, C₁₋₄ alkyl, C₂₋₆alkenyl, C₂₋₆alkynyl, C₃₋₁₀cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl which may be optionally substituted; wherein R₆ may independently or together with R₇ form a C₃-C₁₀ cycloalkyl, C₃₋₁₀ heterocycloalkyl, C₃₋₁₀ cycloalkenyl, C₃₋₁₀ heterocycloalkenyl, C₆-C₁₀ aryl or C₅-C₁₀ heteroaryl which may be optionally substituted;
R₂₀ is selected from
C(═O)R₆,
C(═O)OR₆, C(═O)NR₆R₇, C(═O)NR₆C(═O)R₇, C(═O)C(═O)R₆, C(═O)C(═O)OR₆,
C(═O)C(═O)NR₆R₇, C(═O)NR₇S(═O)OR₆, C(═O)NR₆OR₇, (C═O)SR₆,
S(═O)R₆, S(═O)₂R₆, S(═O)OR₆, S(═O)₂OR₆, S(═O)NR₆R₇, S(═O)₂NR₆R₇,
S(═O)₂NR₇COR₆, S(═O)(═NR₈)NR₆R₇, S(═O)(═NR₆)R₇, S(═NR₆)R₇,
SC(═O)R₆, SC(═O)OR₆, SC(═O)NR₆R₇,
C(═S)R₆, C(═S)OR₆, C(═S)NR₆R₇,
CR₇(═NR₆), CR₇(═N—OR₆), COR₇(═N—OR₆), CNR₇R(═N—OR₆), CR₆(═N—NR₇R₆);
m is an integer selected from 0, 1, 2, 3 or 4;
n is an integer selected from 1 or 2.

In this invention, the optional substituents may be selected from CN (cyano), nitro (NO₂), halogen, OR₆, SR₆, NR₆R₇, NR₆OR₇, NR₆NR₇R₈, R₂₀, OR₂₀, SR₂₀, NR₆R₂₀, C₁₋₆ alkyl, C₃₋₁₀cycloalkyl, C₃₋₁₀ heterocycloalkyl, C₃₋₁₀ cycloalkenyl, C₃₋₁₀ heterocycloalkenyl, C₃₋₁₀ C₆₋₂₀ aryl, C₅₋₂₀ heteroaryl, C₂-C₆ alkenyl and C₂₋₆ alkynyl which may themselves be optionally substituted.

In this invention, where substituents are said to “include” certain groups, said groups are encompassed but not limiting.

Preferred optional substituents are selected from halo, cyano, nitro, OH, C₁₋₄ alkyl, C₁₋₄ haloalkyl, C₁₋₄ alkoxy, C₁₋₄ carboxy, C₂₋₃ alkenyl, C₂₋₃ alkynyl, C₆₋₂₀ aryl, and C₅₋₂₀ heteroaryl.

There may be more than one optional substituent. For instance, there may be one, two or three optional substituents.

Group R₁ is a substituent on the aryl ring on the left-hand side of the molecule as drawn in Formula (I). Z is selected from CH, CR₁ and N. In one embodiment, the group Z is carbon, i.e. the ring is a phenyl ring.

m is an integer between 0 and 4, i.e. may be 0, 1, 2, 3 or 4. In one embodiment, m is 1, i.e. there is one substituent on the ring. This is preferably in the para-position on the ring.

In a further preferred embodiment, m is 2, i.e. there are two substituents on the ring. These are preferably in the ortho- and para-positions on the ring.

Group R₁ may be selected from a variety of substituents which include C₁₋₆ alkyl, cyano, nitro, halogen, R₂₀, OR₆, SR₆, NR₆R₇, NR₆OR₇, NR₆NR₇R₈, R₂₀, OR₂₀, SR₂₀ and NR₆R₂₀. R₁ may be optionally substituted. Optional substituents may be chosen from those groups listed above.

In a preferred embodiment, R₁ may be selected from CN, NH₂, NR₆R₆, NO₂, C₁₋₆ alkoxy, C₁₋₆ carboxy, sulphoxy, halogen, C₁₋₆ alkyl, C₁₋₆ haloalkyl or C₃₋₁₀ heterocyclyl which where appropriate may be optionally substituted, wherein the C₁₋₆ alkyl is preferably substituted with halogen and wherein the halogen is preferably selected from F, C₁ or Br.

The sulphoxy group, for instance, corresponds to R₁ being R₂₀, wherein R₂₀ is S(═O)₂R₆.

In a preferred embodiment, R₁ is optionally substituted with halogen, CN, C₁₋₄ alkyl, C₁₋₄ haloalkyl, C₂₋₃ alkenyl, NH₂, NR₆R₇, C₁₋₄ alkoxy, C(═O)R₆ or C(═O)OR₆, where preferably R₆ and R₇ are selected from H, C₁₋₄ alkyl and C₁₋₄ haloalkyl.

When the R₁ group is a C₁₋₆ alkyl, the C₁₋₆ alkyl group may be optionally substituted with one, two or three substituents. The optional substituents may be halo groups. Such alkyl groups substituted with halo are hereinafter referred to as “haloakyl” groups.

Preferred C₁₋₆ alkyl groups include methyl, ethyl, propyl and butyl, and their isomers, for instance i-propyl and t-butyl. A particularly preferred subset of C₁₋₆ alkyl groups are C₁₋₄ alkyl groups.

Preferred C₁₋₄ alkoxy groups include (but are not limited to) —OMe and —OEt. It is understood that other alkoxy groups, such as C₁₋₆ alkoxy groups, are obtainable through substitution of C₁₋₆ alkyl groups with for example OR₆ or for example when R₁═OR₆.

Preferred C(═O)R₆ (carboxy) groups include C(═O)Me and C(═O)Et.

Particularly preferred groups for R₁ include halogen groups, particularly Cl, F and Br. There may be multiple halogen substituents on the phenyl ring. Substitution on the phenyl ring can occur at any position, e.g. at the ortho, meta and para positions. For example, there may be one halogen substituent on the phenyl ring. Alternatively, there may be two halogen substituents on the phenyl ring. There may also be three halogen substituents on the phenyl ring. It is also possible for four halogen substituents to be present on the phenyl ring. In a preferred embodiment, there are two halogen substituents on the phenyl ring. In a particularly preferred embodiment, the two halogen substituents on the phenyl ring are C₁ and F. In a further preferred embodiment, the two halogen substituents adopt para- and ortho-positions on the phenyl ring. In a particularly preferred further embodiment, the halogen substituent adopting the para-position is F.

Group W is selected from —(C(R₃)₂)ₚ— wherein p is 0, 1, 2 or 3. Preferably p is 0. In a further preferred embodiment, group W is —(CH₂)ₚ— where p is 0, 1, 2 or 3.

R₂ is selected from H or C₁-C₄ alkyl.

R₃ is selected from H and optionally substituted C₁-C₄ alkyl.

R₄ is independently selected from the same groups as listed for R₁. Accordingly, R₄ is selected from CN, nitro, halogen, OR₆, SR₆, NR₆R₇, NR₆OR₇, NR₆NR₇R₈, ONR₆R₇, ON(═CR₆), R₂₀, OR₂₀, SR₂₀, and NR₆R₂₀; C₁₋₆ alkyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, C₂-C₆ alkenyl and C₂₋₆ alkynyl, which may be optionally substituted.

In a preferred embodiment, R₄ may be selected from halogen, NH₂, NR₆R₇, SR₆, C₁₋₆ alkyl, C₁₋₆ haloalkyl, C₃-C₁₀ cycloalkyl, C₆₋₂₀ aryl, C₅₋₂₀ heteroaryl, C₁₋₆alkoxy, C₁₋₆carboxy and C₃₋₁₀ heterocyclyl. R₄ may be optionally substituted. In a preferred embodiment, R₄ is optionally substituted with, for example, halogen, CN, C₁₋₄ alkyl, C₁₋₄ haloalkyl, C₂₋₃ alkenyl, NH₂, NR₆R₇, C₁₋₄ alkoxy, C(═O)R₆ or C(═O)OR₆, where preferably R₆ and R₇ are selected from H and C₁₋₄ alkyl which may themselves be optionally substituted with a group such as halo or hydroxyl. In a preferred embodiment, R₄ is C₃₋₁₀ cycloalkyl which is optionally substituted. In a preferred embodiment R₄ is C₃₋₈ cycloalkyl or C₃₋₈ cycloalkyl.

Preferred C₁₋₆ alkyl groups for R₄ include methyl, ethyl, propyl and butyl, and their isomers, for instance i-propyl and t-butyl. Also preferred are cycloalkyl groups such as cyclopropyl and cyclobutyl. Other preferred groups for R₄ include 5 and 6 membered heterocyclic rings including N, which may be fully or partially saturated, or aromatic. For instance, R₄ may be a phenyl or a pyridine ring.

Preferred groups for R₄ include C₁₋₄ alkyl optionally substituted with halo, otherwise referred to as C₁₋₄ haloalkyl. Other preferred groups for R₄ include optionally substituted C₃₋₁₀ cycloalkyl, C₆-C₁₀ aryl and optionally substituted C₃₋₁₀ heterocyclyl. Where the optional substitution includes halogen, there may be up to three halogen substituents. For instance, a particularly preferred group for R₄ is CF₃ or CF₂H.

Preferred alkyl groups include methyl and ethyl. The R₄ group may alternatively be tert-butyl. In a further embodiment, R₄ is selected from a C₃₋₁₀ cycloalkyl, preferably a C₃₋₈ cycloalkyl, most preferably cyclopropyl or cyclobutyl. In a further embodiment, R₄ is selected from alkoxy (OR₆), ether (—R₆OR₆) ester (OC(═O)OR₆), and carboxylic acid OC(═O)OH, where these may be optionally substituted.

R₄ may be a C₁₋₆ alkyl group substituted with an alkoxy group, for instance, a C₁₋₄ alkoxy group. For instance, group R₄ may be CH₂OCH₃. Preferred ether groups may be represented by —(CH₂)ₙ—OR₈ wherein n is an integer in the range 1-4.

Preferred alkoxy groups include —OMe and —OEt.

Preferred carboxy groups, C(═O)R₈, include C(═O)Me and C(═O)Et.

Preferred amine groups for R₄ include NH₂ and NR₆R₇ wherein R₆ and R₇ are preferably selected from H and C₁₋₄ alkyl.

Group R₂₀ is selected from C(═O)R₆, C(═O)OR₆, C(═O)NR₆R₇, C(═O)NR₆C(═O)R₇, C(═O)C(═O)R₆, C(═O)C(═O)OR₆, C(═O)C(═O)NR₆R₇, C(═O)NR₇S(═O)OR₆, C(═O)NR₆OR₇, (C═O)SR₆, S(═O)R₆, S(═O)₂R₆, S(═O)OR₆, S(═O)₂OR₆, S(═O)NR₆R₇, S(═O)₂NR₆R₇, S(═O)₂NR₇COR₆, S(═O)(═NR₆)NR₆R₇, S(═O)(═NR₆)R₇, S(═NR₆)R₇, SC(═O)R₆, SC(═O)OR₆, SC(═O)NR₆R₇, ONR₆R₇, ON(═CR₆), C(═S)R₈, C(═S)OR₆, C(═S)NR₆R₇, CR₇(═NR₆), CR₇(═N—OR₆), COR₇(═N—OR₈), CNR₇R₈(═N—OR₆), CR₈(═N—NR₇R₆).

Accordingly, the group R₂₀ is selected, along with the groups R₆, R₇ and R₈ where appropriate to give the preferred compounds outlined above.

Accordingly, preferred groups for R₂₀ include C(═O)R₆, C(═O)OR₆, C(═O)NR₆R₇, and S(═O)R₆.

Preferred groups for R₆ and R₇ and R₈ include H, C₁₋₄ alkyl and C₁₋₄ haloalkyl.

The groups R₁ and R₄ (featuring R₂₀ where appropriate) may be chosen such that they include an ether, alkoxyamine, oxime, ester, carbonate, carbamate, sulphite, sulphide, sulphinyl, sulphonyl, sulphinic acid, sulphinamide, sulphonamide, sulphonimidamides, sulphilimine, sulphoximine, sulphenamide, thiolester, thiocarbonate, thiocarbamate, ketone, amide, imide, diketone, ketoacid, ketoamide, acetamide, thioaldehyde, thionoester, thioamide, imine, carboximidate, enamine, azo, nitrile, isonitrile, cyanate or isocyanate.

The right-hand side of the molecule in Formula (I) comprises a 5-membered heterocycle. Such heterocycles comprise multiple isomers and tautomers. Such a heterocycle according to the present invention may be in the form of a (where * denotes the remainder of the molecule, not shown for clarity):
Tetrazole (all four X groups are N):

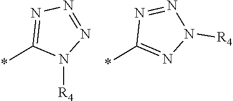

Triazole (three of the four 4 X groups are N):
1,2,4-triazole

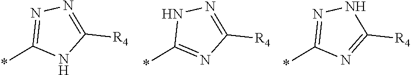

1,2,3-triazole

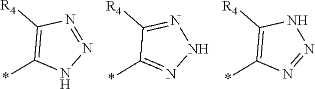

Oxadiazole (one of the X groups is O, and two are N):

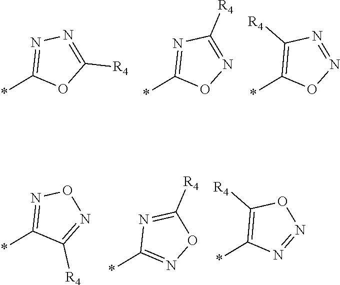

The 5-membered heterocycle is always at least partially substituted, i.e. there is always at least one substituent R₄. In a preferred embodiment, the 5-membered heterocycle is substituted with one R₄ group. In a preferred embodiment, the substitution on the heterocycle occurs at the p-position relative to the remainder of the molecule.

In an embodiment, three of X¹-X⁴ in Formula (I) are N, NH, NR₄ or O, such that the ring containing X¹-X⁴ is aromatic. In an embodiment, three of X¹-X⁴ in Formula (I) are selected from N, NH and NR₄, and the remaining X group is selected from CH and CR₄, such that the 5-membered ring including X¹-X⁴ is a triazole, wherein it is understood that when one of the three X¹-X⁴ selected from N, NH and NR₄ is NH, the remaining X group is CR₄.

In an embodiment, two of X¹-X⁴ are selected from N and one of X¹-X⁴ is O, and the remaining X group is CR₄ such that the 5-membered ring including X¹-X⁴ is an oxadiazole.

As detailed above, particularly preferred groups for R₄ include C₁₋₄ alkyl substituted with halo, otherwise referred to as C₁₋₄ haloalkyl. For instance, the group R₄ may be CF₃ or CF₂H. The R₄ group may alternatively be tert-butyl.

The R₄ group may be an aryl, e.g. phenyl or pyridine.

In other embodiments, the group R₄ is an alkyl alkoxy group. In this instance R₄ is alkyl which is substituted with an OR₆ group. For instance R₄ may be of formula (CH₂)ₙ—O—CH₃ wherein n is 1 to 3 and which may be optionally substituted.

Preferably, the optional substituents are selected from halo, hydroxyl, C₁₋₄ alkyl and C₁₋₄ haloalkyl.

Preferred compounds of the invention are triazoles, i.e. the 5 membered heterocycle on the right hand side of the compound is a triazole. A preferred compound of the invention has Formula (II):

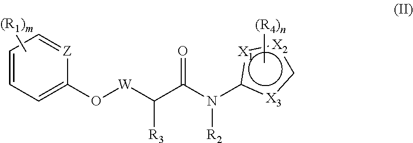

wherein the groups and integers m and n are as defined above for the first aspect of the invention.
X¹-X³ are independently selected from N, NH and NR₄;
and two of X¹-X³ are N
such that the ring containing X¹-X³ is aromatic.

A particularly preferred subgroup of compounds of Formula (II) have the Formula (III):

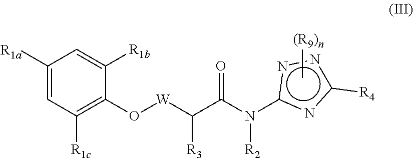

wherein the groups R₃, R₂, W, and R₄ are as defined above for compound (II). R₉ is independently selected from hydrogen, C₁₋₄ alkyl or C₃₋₆ cycloalkyl; and n is 1. The groups R₁ₐ, R_{1b} and R_{1c} are selected from the groups given for R₁ above.

In one embodiment, R₁ₐ, R_{1b} and R_{1c} are independently selected from hydrogen, halide, CN, NO₂, SO₂R₆, SR₆, C₁₋₆ alkyl, C₁₋₆ haloalkyl, C₁₋₆ alkoxy, C₁₋₆ carboxy, NR₆R₇ which may be optionally substituted.

In these preferred embodiments, R₉ is selected from hydrogen, C₁₋₄ alkyl or C₃₋₆ cycloalkyl;

n is equal to 1.

R₄ is preferably a C₁₋₆ alkyl substituted with halide, i.e. C₁₋₆ haloalkyl, preferably wherein the halide is fluoride.

R₁ₐ and R_{1b} are preferably methyl or halide groups, preferably halide groups.

R_{1c} is preferably hydrogen.

In a particularly preferred embodiment, R₁ₐ is chloride or fluoride and R_{1b} is chloride.

R₆ is preferably selected from C₁₋₄ alkyl and C₁₋₄ haloalkyl.

A particularly preferred subgroup of compounds of Formula (III) have a formula selected from the following formulae:

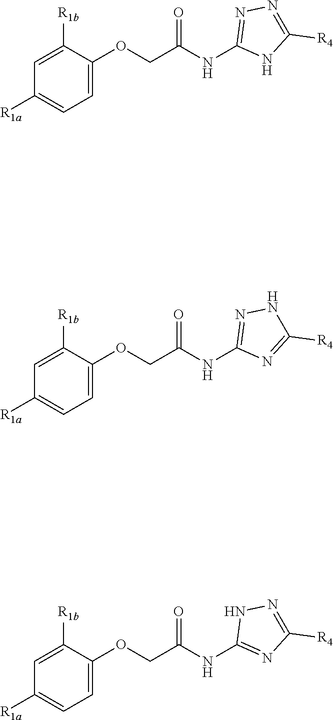

wherein the group R₄ is as defined for the compound of formula II above, and R₁ₐ and R_{1b} are selected from the groups given for R₁.

In a preferred embodiment, R₁ₐ and R_{1b} are independently selected from hydrogen, halide, CN, NO₂, SO₂R₆, SR₆, C₁₋₆ alkyl, C₁₋₆ haloalkyl, C₁₋₆ alkoxy, C₁₋₆ carboxy and NR₆R₇.

In further preferred embodiments, R₄ is a C₁₋₄ haloalkyl, preferably wherein the halide is fluoride.

R₁ₐ and R_{1b} are halide.

R₁ₐ is preferably fluoride and R_{1b} is preferably chloride.

R₆ is preferably selected from C₁₋₄ alkyl and C₁₋₄ haloalkyl.

In a further embodiment of the invention, the compounds of formula I are tetrazoles, i.e. the 5-membered heterocycle on the right-hand side of the compound as drawn above is a tetrazole.

A preferred tetrazole compound has the Formula (IV):

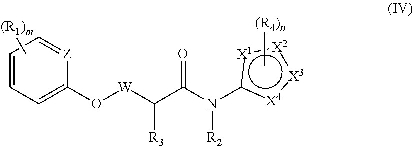

wherein the groups R₁, W, R₂, R₃ and R₄ and integer m are as defined in the first aspect of the invention.
X¹-X⁴ are independently selected from N, NH and NR₄;
wherein three of X¹-X⁴ are N and n is selected from 0 or 1.

For instance, the compound may have Formula (V):

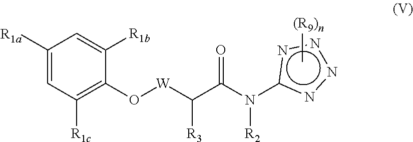

wherein the groups W, R₂ and R₃ are as defined in the first aspect of the invention. Groups R₁ₐ, R_{1b} and R_{1c} are selected from those listed for group R₁ in the first aspect of the invention. R₉ is typically selected from the groups listed for R₄ in the first aspect of the invention. In Formula (V), n is 1.

In a preferred embodiment, R₁ₐ, R_{1b} and R_{1c} are independently selected from hydrogen, halide, CN, NO₂, SO₂R₆, SR₆, C₁₋₆ alkyl, C₁₋₆ haloalkyl, C₁₋₆ alkoxy, C₁₋₆ carboxy and NR₆R₇. R₆ and R₇ are as defined according to the first aspect of the invention and are preferably selected from H, C₁₋₄ alkyl and C₁₋₄ haloalkyl;

R₉ is selected from C₁₋₄ alkyl;
n is 1.

In compounds of Formula (V), preferably R₁ₐ and R_{1b} are halide.

Preferably R_{1c} is hydrogen.

Preferably R₁ₐ is chloride or fluoride and R_{1b} is chloride.

A further preferred tetrazole has the formula:

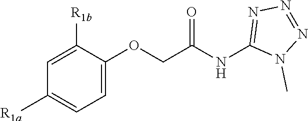

wherein
R₁ₐ and R_{1b} are independently selected from hydrogen, halide, CN, NO₂, SO₂R₆, SR₆, C₁₋₆ alkyl, C₁₋₆ haloalkyl, C₁₋₆ alkoxy, C₁₋₆ carboxy, NR₆R₇; where
R₆ and R₇ are as defined according to the first aspect of the invention and are preferably selected from H, C₁₋₄ alkyl and C₁₋₄ haloalkyl.

Further preferred compounds of the invention are oxadiazoles, i.e. the 5 membered heterocycle on the right hand side of the compound as drawn is an oxadiazole. N- and O-containing 5-membered heterocyclic rings may have different isomeric forms. Preferred compounds may have Formula (VI):

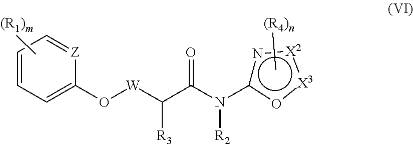

wherein R₁, W, R₂, R₃, R₄, m and n are as defined in the first aspect of the invention;
X²-X³ are independently selected from N and CR₄;
wherein one of X²-X³ is N.

Alternatively, the formula may be (VII):

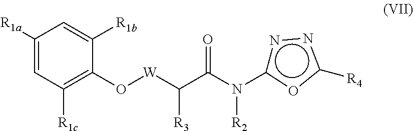

wherein W, R₂, R₃ and R₄ are as defined in the first aspect of the invention. R₁ₐ, R_{1b} and R_{1c} are selected from the groups given for R₁ in the first aspect of the invention.

In preferred compounds of formula VII, R₁ₐ, R_{1b} and R_{1c} are independently selected from hydrogen, halide, CN, NO₂, SO₂R₆, SR₆, C₁₋₆ alkyl, C₁₋₆ haloalkyl, C₁₋₆ alkoxy, C₁₋₆ carboxy, NR₆R₇;

R₄ is as defined according to the first aspect of the invention;
R₆ and R₇ are as defined according to the first aspect of the invention and are preferably selected from H, C₁₋₄ alkyl and C₁₋₄ haloalkyl.

Alternatively, the compound may have Formula (VIII):

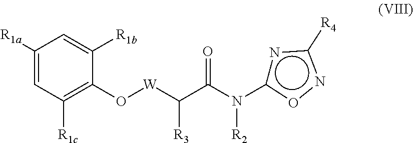

wherein W, R₂, R₃ and R₄ are as defined in the first aspect of the invention. R₁ₐ, R_{1b} and R_{1c} are selected from the groups given for R₁ in the first aspect of the invention.

In preferred compounds of formula (VIII), R₁ₐ, R_{1b} and R_{1c} are independently selected from hydrogen, halide, CN, NO₂, SO₂R₆, SR₆, C₁₋₆ alkyl, C₁₋₆ haloalkyl, C₁₋₆ alkoxy, C₁₋₆ carboxy, NR₆R₇;

R₄ is as defined according to the first aspect of the invention;
R₆ and R₇ are as defined according to the first aspect of the invention.

In preferred compounds, R₄ is a C₁₋₄ haloalkyl, preferably wherein the halide is fluoride;

R₁ₐ and R_{1b} are halide;
Even more preferably R₁ₐ is chloride or fluoride and R_{1b} is chloride.

A further preferred subgroup of compounds of Formula (VII) have the formula:

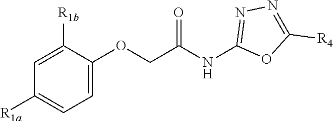

wherein
R₁ₐ and R_{1b} are independently selected from hydrogen, halide, CN, NO₂, SO₂R₆, SR₆, C₁₋₆ alkyl, C₁₋₆ haloalkyl, C₁₋₆ alkoxy, C₁₋₆ carboxy, NR₆R₇;
R₄ is defined as defined according to the first aspect of the invention.

In preferred compounds, R₄ is an alkyl substituted with halide, preferably wherein the halide is fluoride;

preferably R₁ₐ and R_{1b} are halide;
preferably R₁ₐ is chloride or fluoride and R_{1b} is chloride.

A further preferred subgroup of compounds of Formula (VIII) have the formula:

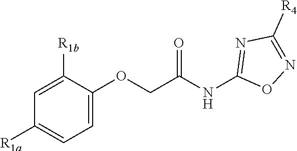

wherein
R₁ₐ and R_{1b} are independently selected from hydrogen, halide, CN, NO₂, SO₂R₆, SR₆, C₁₋₆ alkyl, C₁₋₆ haloalkyl, C₁₋₆ alkoxy, C₁₋₆ carboxy, NR₆R₇;
R₄ is defined as defined according to the first aspect of the invention.

In preferred compounds, R₄ is an alkyl substituted with halide, preferably wherein the halide is fluoride;

preferably R₁ₐ and R_{1b} are halide;
preferably R₁ₐ is chloride or fluoride and R_{1b} is chloride.

### Definitions of Groups

Where denoted in the claims, substituents and functional groups may be optionally substituted. It may manifest in single or multiple substitution, and can include different tautomers, isomers and stereoisomers where appropriate.

Alkyl groups (e.g. C₁-C₆ alkyl) can include methyl (Me), ethyl (Et), n-propyl (n-Pr), isopropyl (i-Pr), n-butyl (n-Bu), isobutyl (i-Bu), sec-butyl (s-Bu) and tert-butyl (t-Bu). Alkyl groups are generally C₁-C₆ alkyl and are preferably C₁-C₄ alkyl.

Cycloalkyl groups (e.g. C₃-C₁₀ cycloalkyl) include, for example cyclopropyl (c-propyl, c-Pr), cyclobutyl (c-butyl, c-Bu), cyclopentyl (c-pentyl) and cyclohexyl (c-hexyl). Cycloalkyl groups are generally C₃-C₁₀.

Alkenyl and alkynyl moieties may exist as straight or branched chains. Alkenyl moieties may be of either (E)- or (Z)-configuration where appropriate. These include vinyl, allyl and propargyl, for example. Alkenyl and alkynyl moieties can contain multiple double and triple bonds in any combination. For example, an alkenyl moiety could contain two separate alkene double bonds, or one double bond and a triple bond. Alkenyl and alkynyl groups are generally C₂-C₆ and are preferably C₂-C₄.

Cycloalkenyl groups (e.g. C₃₋₁₀ cycloalkenyl), also known as cycloolefins, include, for example cyclopropylene, cyclobutylene, cyclopentylene and cyclohexylene. The cycloalkenyl groups of the present invention are preferably at least C₄ in ring size, preferably C₅ and above.

Cycloalkenyl moieties can contain multiple double bonds in any combination. For example, a cycloalkenyl moiety could contain two separate alkene double bonds.

Halogen (which may be written halo or halide) includes fluorine, chlorine, bromine or iodine. This definition of halogen further applies in other situations, for example haloalkyl, haloaryl or haloalkenyl. For example, haloalkyl includes bromoethyl, fluoroethyl; haloaryl includes bromobenzyl, fluorobenzyl; and haloalkenyl includes ethylene dibromide or ethylene difluoride.

Haloalkyl groups (e.g. C₁-C₆ haloalkyl) are, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl, heptafluoro-n-propyl and perfluoro-n-hexyl.

Alkoxy groups (e.g. C₁-C₄ alkoxy) include for example methoxy, ethoxy, propoxy, isopropoxy, n-butoxy, isobutoxy, sec-butoxy or tert-butoxy.

Alkoxyalkyl groups (e.g. C₁-C₆ alkoxy-C₁-C₃ alkyl) include for example methoxymethyl, methoxyethyl, ethoxymethyl, ethoxyethyl, n-propoxymethyl, n-propoxyethyl, isopropoxymethyl or isopropoxyethyl.

C₁-C₆ alkyl-S-(thioalkyl) is, for example, methylthio, ethylthio, propylthio, isopropylthio, n-butylthio, isobutylthio, sec-butylthio or tert-butylthio.

C₁-C₆ alkyl-S(O)— (alkylsulfinyl) is, for example, methylsulfinyl, ethylsulfinyl, propylsulfinyl, isopropylsulfinyl, n-butylsulfinyl, isobutylsulfinyl, sec-butylsulfinyl or tert-butylsulfinyl.

C₁-C₆ alkyl-S(O)₂— (alkylsulfonyl) is, for example, methylsulfonyl, ethylsulfonyl, propylsulfonyl, isopropylsulfonyl, n-butylsulfonyl, isobutylsulfonyl, sec-butylsulfonyl or tert-butylsulfonyl.

Heterocyclyl, where not otherwise stated, is a ring structure which may be aromatic or fully or partially saturated and may contain from 1 to 4 heteroatoms each independently selected from the group consisting of oxygen, nitrogen and sulphur. The term “heterocyclyl” encompasses heterocycloalkyl, heterocycloalkenyl and heteroaryl.

The terms heterocycloalkyl and heterocycloalkenyl denote structural equivalents to cycloalkyl and cycloalkenyl which may contain from 1 to 4 heteroatoms each independently selected from the group consisting of oxygen, nitrogen and sulphur, and which are typically not aromatic. Heterocycloalkenyl rings can contain multiple double bonds in any combination.

The term aryl denotes an aromatic hydrocarbon, which for example includes, phenyl, tolyl, xylyl and naphthyl groups. Aryl groups may be singly or multiply substituted at different positions around the ring. In this invention, aryl groups are generally C₆-C₂₀ and are preferably C₆-C₁₀.

The term heteroaryl denotes an aryl group in which at least one atom in the aromatic ring is a heteroatom, such as S (e.g. thiophene), O (e.g. furan), or N (e.g. indole). Heteroaryl groups may have more than one heteroatom in the ring (for example cytosine). In this invention, heteroaryl groups are generally C₅-C₂₀ and are preferably C₅-C₁₀.

In addition, the present invention concerns agriculturally acceptable salts of compounds of Formula (I). These salts may include those capable of being formed by reaction with amines, bases of Group 1 and Group 2 elements or quaternary ammonium bases. Of particular interest in the bases of Group 1 and Group 2 elements are those comprising hydroxides of Li, Na, K, Mg and Ca, of which NaOH and KOH are the most important. The compounds of Formula (I) according to the invention may also include hydrates generated during salt formation.

Amines suitable for ammonium salt formation include ammonia, primary, secondary and tertiary C₁-C₁₈ alkylamines, C₁-C₄ hydroxyalkylamines and C₂-C₄ alkoxyalkylamines. The person skilled in the art would be aware of which amines would be suitable for the formation of ammonium salts. Preferred amines for ammonium salt formation are triethylamine, isopropylamine and diisopropylamine.

Depending on the nature of the substituents, compounds of Formula (I) may exist in different isomeric forms. When X¹-X⁴ are chosen such that the heterocyclic ring is a triazole, for example, compounds of Formula (I) may exist in different tautomeric forms.

This invention covers all isomers, tautomers and mixtures thereof in any and all proportions. Where double bonds are present, cis- and trans-isomers can exist. Such isomers fall within the scope of the present invention. Compounds of Formula (I) may contain stereocentres and may exist as a single enantiomer, pairs of enantiomers in any proportion or, where more than one stereocentre is present, contain diastereoisomers in all possible ratios. In general, one of the enantiomers has enhanced biological activity compared to the other possibilities.

### Agricultural Use

Compounds of the invention are used as agrochemicals, or agricultural chemicals, which are those chemicals used for agricultural purposes. Agrochemicals are classified based on the role for which they are being used, e.g. pesticides for the controlling of pests, fungicides for the controlling of fungal growth, fertilisers for enhancing the nutrient content of the soil in which crops are grown, or herbicides, which are used to destroy unwanted vegetation. Compounds according to the present invention may be used as any agrichemical, but are preferably used as herbicides.

Herbicides in particular may be selective or non-selective. The former are herbicides designed for use around desired plants/crops and seek to control weeds without damaging the desired plant/crop itself. The latter are herbicides which do not discriminate on variety of plant, but instead destroy all vegetation. Compounds according to the present invention are preferably selective herbicides.

Herbicides may be applied by a variety of routes and may have a variety of mechanisms of action. They can be applied to the soil, so as to be absorbed by the roots/shoots of emerging weed seedlings, or they can be applied to the leaves of existing plants. The choice of route can also dictate whether a herbicide is a pre-emergence herbicide (i.e. applied before the weed seedlings emerge at the surface) or a post-emergence herbicide (one which is applied after the weed seedlings have emerged through the soil surface). Compounds and compositions according to the present invention may be used as pre-emergence and post-emergence herbicides. Preferably these compounds and compositions are used as post-emergence herbicides.

The compounds of the invention may be used as herbicides in isolation, but are typically formulated into agrochemical compositions, preferably herbicidal compositions, using formulation adjuvants, such as carriers, solvents and surface-active agents (SFAs or called surfactants). In an aspect of the present invention is provided a herbicidal composition comprising a herbicidal compound according to Formula (I) and an agriculturally acceptable excipient. The relevant agrochemical composition can be in the form of concentrates, requiring dilution prior to use, or may be formulated for immediate application without further processing. Dilution prior to use most typically involves water but can also be undertaken with substances other than water, such as liquid fertilisers, micronutrients, biological organisms, oil or solvents, or may be made with water and one or more of said substances in conjunction.

Any references to compounds of Formula (I) include reference to the specific embodiments of Formula (II)-(VIII).

The herbicidal compositions generally contain from 0.1-99% w/w of compounds according to Formula (I) and 1-99.9% w/w of an excipient, preferably including 0-25% w/w of a surfactant.

The formulation used may be chosen from a range of formulation classes, the details of which are known from the Manual on Development and Use of FAO Specifications for Plant Protection Products, 5th Edition, 1999, and subsequent related documents. These include dustable powders (DP), soluble powders (SP), wettable powders (WP), water soluble granules (SG), water dispersible granules (WG), granules (GR) (slow or quick release), soluble concentrates (SL), oil miscible liquids (OL), ultra-low volume liquids (UL), dispersible concentrates (DC), emulsifiable concentrates (EC), emulsions (both oil in water (EW) and water in oil (EO)), micro-emulsions (ME), suspension concentrates (SC), capsule suspensions (CS), aerosols and seed treatment formulations. The chosen formulation in question will depend upon the particular desired purpose and end use of the formulation and the physical, chemical and biological properties of the compound of Formula (I).

The composition may include one or more additives to enhance the biological performance of the composition, for example by improving wetting, retention or distribution on and across surfaces; fastness on treated surfaces in the presence of rain or other naturally occurring water source (e.g. dew); or uptake or mobility of a compound of Formula (I). Such additives include surfactants, spray additives based on oils, for example certain mineral oils or natural plant oils (such as soy bean and rape seed oil), and blends of these with other bioenhancing adjuvants (ingredients which may aid or modify the action of a compound of Formula (I)).

Suitable suspending agents include hydrophilic colloids (such as polysaccharides, polyvinylpyrrolidone or sodium carboxymethylcellulose) and swelling clays (such as bentonite or attapulgite).

Wetting agents, dispersing agents and emulsifying agents may be surfactants of the cationic, anionic, amphoteric or non-ionic type.

Suitable cationic surfactants include quaternary ammonium compounds (for example cetyltrimethyl ammonium bromide), imidazolines and amine salts.

Suitable anionic surfactants include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-Isopropyl- and tri-Isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (for producing monoesters) or phosphorus pentoxide (for producing diesters), e.g. the reaction between dodecanol and tetraphosphoric acid; furthermore these products may be alkoxylated, generally ethoxylated), sulphosuccinamates, paraffin or olefin sulphonates, taurates and lignosulphonates.

Suitable amphoteric surfactants include betaines, propionates and glycinates.

Suitable non-ionic surfactants include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

The composition of the present may further comprise at least one additional pesticide. For example, the compounds according to the invention can also be used in conjunction with other herbicides, pesticides or plant growth regulators. The additional pesticide is preferably a herbicide and/or herbicide safener. Examples of said mixtures are (in which I indicates a compound according to Formula (I)): I+acetochlor, I+acifluorfen, I+acifluorfen-sodium, I+aclonifen, I+acrolein, I+alachlor, I+alloxydim, I+ametryn, I+amicarbazone, I+amidosulfuron, I+aminopyralid, I+amitrole, I+anilofos, I+asulam, I+atrazine, I+azafenidin, I+azimsulfuron, I+BCPC, I+beflubutamid, I+benazolin, I+bencarbazone, I+benfluralin, I+benfuresate, I+bensulfuron, I+bensulfuron-methyl, I+bensulide, I+bentazone, I+benzfendizone, I+benzobicyclon, I+benzofenap, I+bicyclopyrone, I+bifenox, I+bilanafos, I+bispyribac, I+bispyribac-sodium, I+borax, I+bromacil, I+bromobutide, I+bromoxynil, I+butachlor, I+butamifos, I+butralin, I+butroxydim, I+butylate, I+cacodylic acid, I+calcium chlorate, I+cafenstrole, I+carbetamide, I+carfentrazone, I+carfentrazone-ethyl, I+chlorflurenol, I+chlorflurenol-methyl, I+chloridazon, I+chlorimuron, I+chlorimuron-ethyl, I+chloroacetic acid, I+chlorotoluron, I+chlorpropham, I+chlorsulfuron, I+chlorthal, I+chlorthal-dimethyl, I+cinidon-ethyl, I+cinmethylin, I+cinosulfuron, I+cisanilide, I+clethodim, I+clodinafop, I+clodinafop-propargyl, I+clomazone, I+clomeprop, I+clopyralid, I+cloransulam, I+cloransulam-methyl, I+cyanazine, I+cycloate, I+cyclopyranile, I+cyclosulfamuron, I+cycloxydim, I+cyhalofop, I+cyhalofop-butyl, I+2,4-D, I+daimuron, I+dalapon, I+dazomet, I+2,4-DB, I+desmedipham, I+dicamba, I+dichlobenil, I+dichlorprop, I+dichlorprop-P, I+diclofop, I+diclofop-methyl, I+diclosulam, I+difenzoquat, I+difenzoquat metilsulfate, I+diflufenican, I+diflufenzopyr, I+dimefuron, I+dimepiperate, I+dimethachlor, I+dimethametryn, I+dimethenamid, I+dimethenamid-P, I+dimethipin, I+dimethylarsinic acid, I+dinitramine, I+dinoterb, I+diphenamid, I+dipropetryn, I+diquat, I+diquat dibromide, I+dithiopyr, I+diuron, I+endothal, I+EPTC, I+esprocarb, I+ethalfluralin, I+ethametsulfuron, I+ethametsulfuron-methyl, I+ethephon, I+ethofumesate, I+ethoxyfen, I+ethoxysulfuron, I+etobenzanid, I+fenoxaprop-P, I+fenoxaprop-P-ethyl, I+fenquinotrione, I+fentrazamide, I+ferrous sulfate, I+flamprop-M, I+flazasulfuron, I+florpyrauxifen, I+florasulam, I+fluazifop, I+fluazifop-butyl, I+fluazifop-P, I+fluazifop-P-butyl, I+fluazolate, I+flucarbazone, I+flucarbazone-sodium, I+flucetosulfuron, I+fluchloralin, I+flufenacet, I+flufenpyr, I+flufenpyr-ethyl, I+flumetralin, I+flumetsulam, I+flumiclorac, I+flumiclorac-pentyl, I+flumioxazin, I+flumipropin, I+fluometuron, I+fluoroglycofen, I+fluoroglycofenethyl, I+fluoxaprop, I+flupoxam, I+flupropacil, I+flupropanate, I+flupyrsulfuron, I+flupyrsulfuron-methyl-sodium, I+flurenol, I+fluridone, I+flurochloridone, I+fluroxypyr, I+flurtamone, I+fluthiacet, I+fluthiacet-methyl, I+fomesafen, I+foramsulfuron, I+fosamine, I+glufosinate, I+glufosinate-ammonium, I+glyphosate, I+halauxifen, I+halosulfuron, I+halosulfuron-methyl, I+haloxyfop, I+haloxyfop-P, I+hexazinone, I+imazamethabenz, I+imazamethabenz-methyl, I+imazamox, I+imazapic, I+imazapyr, I+imazaquin, I+imazethapyr, I+imazosulfuron, I+indanofan, I+indaziflam, I+iodomethane, I+iodosulfuron, I+iodosulfuron-methyl-sodium, I+ioxynil, I+isoproturon, I+isouron, I+isoxaben, I+isoxachlortole, I+isoxaflutole, I+isoxapyrifop, I+karbutilate, I+lactofen, I+lenacil, I+linuron, I+mecoprop, I+mecoprop-P, I+mefenacet, I+mefluidide, I+mesosulfuron, I+mesosulfuron-methyl, I+mesotrione, I+metam, I+metamifop, I+metamitron, I+metazachlor, I+methabenzthiazuron, I+methazole, I+methylarsonic acid, I+methyldymron, I+methyl isothiocyanate, I+metolachlor, I+S-metolachlor, I+metosulam, I+metoxuron, I+metribuzin, I+metsulfuron, I+metsulfuron-methyl, I+molinate, I+monolinuron, I+naproanilide, I+napropamide, I+napropamide-M, I+naptalam, I+neburon, I+nicosulfuron, I+n-methyl glyphosate, I+nonanoic acid, I+norflurazon, I+oleic acid (fatty acids), I+orbencarb, I+orthosulfamuron, I+oryzalin, I+oxadiargyl, I+oxadiazon, I+oxasulfuron, I+oxaziclomefone, I+oxyfluorfen, I+paraquat, I+paraquat dichloride, I+pebulate, I+pendimethalin, I+penoxsulam, I+pentachlorophenol, I+pentanochlor, I+pentoxazone, I+pethoxamid, I+phenmedipham, I+picloram, I+picolinafen, I+pinoxaden, I+piperophos, I+pretilachlor, I+primisulfuron, I+primisulfuron-methyl, I+prodiamine, I+profoxydim, I+prohexadione-calcium, I+prometon, I+prometryn, I+propachlor, I+propanil, I+propaquizafop, I+propazine, I+propham, I+propisochlor, I+propoxycarbazone, I+propoxycarbazone-sodium, I+propyzamide, I+prosulfocarb, I+prosulfuron, I+pyraclonil, I+pyraflufen, I+pyraflufen-ethyl, I+pyrasulfotole, I+pyrazolynate, I+pyrazosulfuron, I+pyrazosulfuron-ethyl, I+pyrazoxyfen, I+pyribenzoxim, I+pyributicarb, I+pyridafol, I+pyridate, I+pyriftalid, I+pyriminobac, I+pyriminobac-methyl, I+pyrimisulfan, I+pyrithiobac, I+pyrithiobac-sodium, I+pyroxasulfone, I+pyroxsulam, I+quinclorac, I+quinmerac, I+quinoclamine, I+quizalofop, I+quizalofop-P, I+rimsulfuron, I+saflufenacil, I+sethoxydim, I+siduron, I+simazine, I+simetryn, I+sodium chlorate, I+sulcotrione, I+sulfentrazone, I+sulfometuron, I+sulfometuron-methyl, I+sulfosate, I+sulfosulfuron, I+sulfuric acid, I+tebuthiuron, I+tefuryltrione, I+tembotrione, I+tepraloxydim, I+terbacil, I+terbumeton, I+terbuthylazine, I+terbutryn, I+thenylchlor, I+thiazopyr, I+thifensulfuron, I+thiencarbazone, I+thifensulfuron-methyl, I+thiobencarb, I+tolpyralate, I+topramezone, I+tralkoxydim, I+tri-allate, I+triasulfuron, I+triaziflam, I+tribenuron, I+tribenuronmethyl, I+triclopyr, I+trietazine, I+trifloxysulfuron, I+trifloxysulfuron-sodium, I+trifludimoxazin, I+trifluralin, I+triflusulfuron, I+triflusulfuron-methyl, I+trihydroxytriazine, I+trinexapac-ethyl, I+tritosulfuron, I+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester.

The components mixed with the compound of Formula (I) may also be in the form of esters or salts.

The compound of Formula (I) can also be used in mixtures with other agrochemicals such as fungicides, nematicides or insecticides, examples of which are given in The Pesticide Manual. The mixing ratio of the compound of Formula (I) to the other component is preferably from 1:100 to 1000:1. The mixtures can advantageously be used in the formulations above (in which case “active ingredient” relates to the respective mixture of compound of Formula (I) with the other component).

The compounds of Formula (I) according to the invention can also be used together with one or more herbicide safeners. Similarly, compounds according to Formula (I) can be mixed with one or more further herbicides and may also be used in combination with one or more herbicide safeners. The herbicide safeners can be AD 67 (MON 4660), benoxacor, cloquintocet-mexyl, cyprosulfamide (CAS RN 221667-31-8), dichlormid, fenchlorazole-ethyl, fenclorim, fluxofenim, furilazole and the corresponding R isomer, isoxadifen-ethyl, mefenpyr-diethyl, oxabetrinil, N-isopropyl-4-(2-methoxy-benzo-ylsulfamoyl)-benzamide (CAS RN 221668-34-4), among others. Herbicide safeners can also include compounds disclosed in, for example, EP0365484 e.g. N-(2-methoxybenzoyl)-4-[(methylaminocarbonyl)amino]benzenesulfonamide. The herbicide safeners used with the compound of Formula (I) may also be in the form of esters or salts.

Preferably the mixing ratio of compound of Formula (I) to herbicide safener is from 100:1 to 1:10, especially from 20:1 to 1:1. The mixtures can beneficially be used in the formulations discussed above (in which case “active ingredient” relates to the respective mixture of compound of Formula (I) with the herbicide safener).

The present invention still further provides a method of controlling weeds at a locus comprising crop plants and weeds, wherein the method comprises application to the locus of a weed controlling amount of a composition according to the present invention. ‘Controlling’ in an agrochemical context means killing, reducing or retarding growth or preventing or reducing germination. The plants to be controlled are unwanted plants, i.e. weeds. ‘Locus’ denotes the position or place in which the plants are growing or will grow.

The application rate of compounds of Formula (I) may vary within a significant range and is dependent on the nature and qualities of the soil, the method of application (pre- or post-emergence; seed dressing; application to the seed furrow; no/minimal tillage application etc.), the crop plant, the weed or weeds to be controlled, actual climatic and meteorological conditions, and other factors determined by the method of application. The compounds of Formula (I) according to the invention are typically applied at a rate of from 10 to 2000 g/ha, especially from 50 to 1000 g/ha.

The application is generally made by spraying the composition, typically by tractor mounted sprayer for large areas, but other methods such as dusting (e.g. from airborne delivery mechanisms), drip or drench can also be used among others.

Useful plants, the protection of which is achieved by application of compositions of the present invention, include crops such as cereals, for example barley and wheat, cotton, oilseed rape, sunflower, maize, rice, soybeans, sugar beet, sugar cane and turf. Crop plants can also include trees, such as fruit trees, palm trees, coconut trees or other nuts. Also included are vines such as grapes, fruit bushes, fruit plants, vegetables and legumes.

The term ‘crops’ further includes those crops which have been rendered tolerant to herbicides or classes thereof (e.g. ALS-, GS-, EPSPS-, PPO-, ACCase- and HPPD-inhibitors) by conventional selective breeding or by genetic engineering/modification. An example of a crop that has been rendered tolerant to imidazolinones, e.g. imazamox, by conventional methods of selective breeding is Clearfield® summer rape (canola). Examples of crops that have been rendered tolerant to herbicides by genetic engineering methods include e.g. glyphosate- and glufosinate-resistant maize varieties (known commercially under the trade names RoundupReady® and LibertyLink® respectively). The term crops also encompasses crops which have been developed to improve their resistance to harmful insects by genetic modification, for example Bt maize (resistant to the European corn borer), Bt cotton (resistant to the cotton boll weevil) and also Bt potatoes (resistant to the Colorado beetle). The Bt toxin is a protein formed by Bacillus thuringiensis. Similar toxins, or genetically modified plants capable of synthesising such toxins, are described for example in WO 95/34656 and WO 03/052073. Examples of transgenic plants modified to enhance insecticidal resistance and express one or more toxins like Bt include KnockOut® (maize), Bollgard® (cotton) and NewLeaf® (potatoes).

Other useful plants include turf grass for example in lawns, parks and roadsides, or grown commercially for sod, and ornamental plants such as flowers or bushes. The compositions of the present invention can be used to control weeds. The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium. The compounds of the present invention have been shown to exhibit particularly good activity against certain grass weed species, especially Lolium multiflorum and Echinochloa crus-galli, and flowering weed species, especially Amaranthus retroflexus, Veronica persica. Weeds can further include plants which may in other situations be deemed crop/valuable plants. They include those growing outside a designated crop area (‘escapes’), and those growing from seeds left behind from previous different crops (‘volunteers’). These volunteers or escapes may be tolerant to certain other herbicides, and this tolerance can arise either naturally, through selective breeding or through genetic modification.

### Examples

Compounds of Formula (I) may then be prepared by reacting a compound of Formula (C), where R′═H, with an amine, or suitable amine salt, of Formula (D) using standard literature methods as would be known to someone skilled in the art. For example, the method used by Carlsson et al. (“Structure-Activity Relationships and Molecular Modeling of 1,2,4-Triazoles as Adenosine Receptor Antagonists”, ACS Medicinal Chemistry Letters (2012), 3(9), 715-720).

Compounds of Formula (C), if not commercially available, may be prepared by reacting a compound of Formula (A) with an alkyl halide such as chloride or bromide, of Formula (B) where, for example, R′=ethyl or methyl, using standard literature methods; for example the method used by Kyeong et al. (“(Aryloxyacetylamino)benzoic Acid Analogues: A New Class of Hypoxia-Inducible Factor-1 Inhibitors”, J. Med. Chem., 2007, 50, 7, 1675-1684). Esters of formula (C), for example where R′=ethyl or methyl, may be transformed to carboxylic acids, R′═H, using standard literature methods as would be known to someone skilled in the art. For example, by treatment with lithium hydroxide.

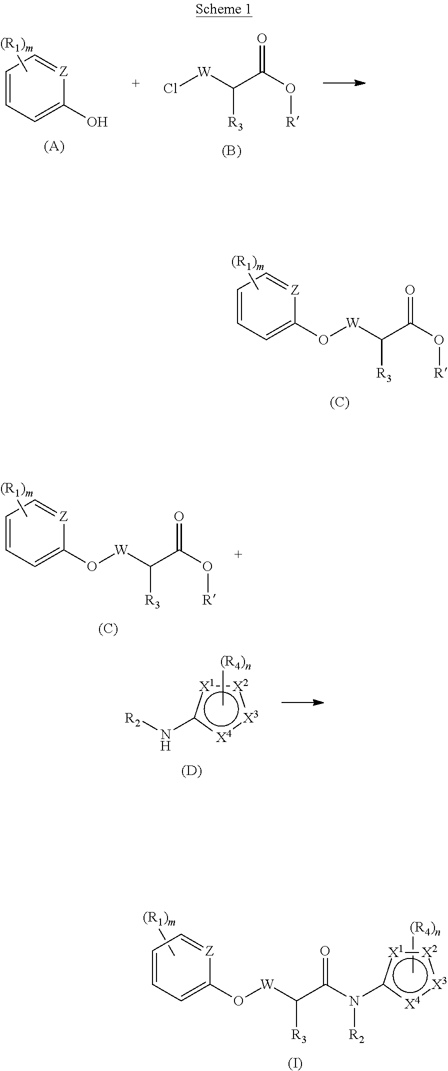

Alternatively, compounds of Formula (I) where one of X¹-X⁴═NR₄ may be prepared from compounds of Formula (I) where the appropriate X═NH using an alkylating agent and suitable base, for example an alkyl iodide and potassium carbonate. For example, compounds where one of X¹-X⁴═NMe can be prepared using a methylating agent and a suitable base, for example methyl iodide and potassium carbonate.

For all specific examples listed below, all starting materials were commercially available or have routes for their synthesis published in the literature of the field. For example, suitable methods are documented in the following publications:

| Compound | Reference |
|---|---|
| 2-(2-chloro-4- | CN 111072615 |
| fluorophenoxy)acetic acid | |
| 2-(4-chloro-3- | WO 2020/012339 |
| fluorophenoxy)acetic acid | |
| 2-(4-chloro-3- | J. Med. Chem., 143, 1826-1839; 2018 |
| fluorophenoxy)acetic acid | |
| 4-(2-chloro-4- | Angew. Chem., Int. Ed., 59(6), |
| fluorophenoxy)butanoic acid | 2429-2439; 2020 |
| 4-(2,4- | J. Agric. Food Chem., 64(37), |
| dichlorophenoxy)butanoic acid | 6911-6915; 2016 |
| 2-(2,4- | WO 2019/179265 |
| dichlorophenoxy)propanoic acid | |
| 2-(2-chloro-4-fluorophenoxy)-N- | Russ. J. Org. Chem., 41(7), |
| [5-(trifluoromethyl)-4H-1,2,4- | 1071-1080; 2005 |
| triazol-3-yl]acetamide | |

### Example 1: 2-(2-chloro-4-fluorophenoxy)-N-[5-(propan-2-yl)-4H-1,2,4-triazol-3-yl]acetamide (23)

2-(2-chloro-4-fluorophenoxy)acetic acid (1.1 mmol) and a solution of N-hydroxybenzotriazole in DMSO (100 g/L, 2 mL, 1.5 mmol) were placed in a vial, and 5-(propan-2-yl)-4H-1,2,4-triazol-3-amine (1 mmol) was added. The reaction mixture was stirred for 30 min in a shaker, and 1-ethyl-3-(3-dimethylaminopropyl)carbodiimide hydrochloride (1.2 mmol) was added. After all the reagents were loaded, the vial was sealed and stirred in a shaker for 1 h. If clear solution was formed, the vial was left at room temperature for 24 h. Otherwise, the reaction mixture was kept in a sonication bath for 24 h (strong heating should be avoided). If strong thickening of the reaction mixture was observed so that stirring was not effective, 0.2 mL of DMSO might be added in one portion. The crude reaction mixture was subjected to HPLC purification to give 2-(2-chloro-4-fluorophenoxy)-N-[5-(propan-2-yl)-4H-1,2,4-triazol-3-yl]acetamide (11%) as a beige solid LC-MS: M+H=313.

### Example 2: 2-(4-chloro-3-fluorophenoxy)-N-[5-(trifluoromethyl)-4H-1,2,4-triazol-3-yl]acetamide (16)

A vial was charged with 2-(4-chloro-3-fluorophenoxy)acetic acid (0.6 mmol) and dry acetonitrile (1 mL). Diisopropylethylamine (1.6 mmol) was added dropwise to the solution. A mixture of 5-(trifluoromethyl)-4H-1,2,4-triazol-3-amine (0.5 mmol) and of 2-chloro-N-methylpyridinium iodide (0.72 mmol) was added with stirring. The reaction vial was placed into a water bath and left at 100° C. for 6 h. Reaction mixture was cooled to room temperature and water was added until the vial is full. Then the vial was sonicated in an ultrasonic bath. The solvent was evaporated. The residue was dissolved in DMSO and filtered. The solution was subjected to HPLC purification to give 2-(4-chloro-3-fluorophenoxy)-N-[5-(trifluoromethyl)-4H-1,2,4-triazol-3-yl]acetamide (45%) as a beige solid LC-MS: M+H=338.

### Example 3: 2-(2,4-difluorophenoxy)-N-[5-(trifluoromethyl)-4H-1,2,4-triazol-3-yl]acetamide (10)

A vial was charged with 2-(2,4-difluorophenoxy)acetic acid (0.1 mmol) and 1,1′-carbonyldiimidazole (0.11 mmol) (as a 15% solution in DMSO). The reaction mixture was heated with stirring for 2 h at 50° C. Then 5-(trifluoromethyl)-4H-1,2,4-triazol-3-amine (0.11 mmol) was added. The vial was sealed and the reaction mixture was heated for 6 h at 100° C. After cooling to ambient temperature the mixture was filtered and the solution was subjected to HPLC purification to give 2-(2,4-difluorophenoxy)-N-[5-(trifluoromethyl)-4H-1,2,4-triazol-3-yl]acetamide (40%) as a yellow solid LC-MS: M+H=323

### Example 4: 4-(2-chloro-4-fluorophenoxy)-N-[5-(trifluoromethyl)-4H-1,2,4-triazol-3-yl]butanamide (30)

Diisopropylethylamine (2.25 mmol), HATU (CAS RN 148893-10-1) (1.875 mmol) and an 3-(trifluoromethyl)-1H-1,2,4-triazol-5-amine (1.65 mmol) were added to a stirred solution of 4-(2-chloro-4-fluorophenoxy)butanoic acid (1.5 mmol) in DCM (0.25 M) at room temperature. The reaction mixture was stirred at room temperature for 18 h. The mixture was partitioned between DCM and H₂O and combined organic extracts were washed with brine, dried over Na₂SO₄ and concentrated. The crude reaction mixture subjected to flash column chromatography (hexane/EtOAc) to give 4-(2-chloro-4-fluorophenoxy)-N-[5-(trifluoromethyl)-4H-1,2,4-triazol-3-yl]butanamide (49%) as a white solid ¹H NMR (600 MHz, DMSO-ds) δ 7.94 (bs, 2H), 7.43-7.38 (m, 1H), 7.16 (dd, J=7.4, 1.9 Hz, 2H), 4.12 (t, J=6.3 Hz, 2H), 3.16 (t, J=7.1 Hz, 2H), 2.12 (q, J=6.7 Hz, 2H).

### Example 5: 4-(2,4-dichlorophenoxy)-N-[5-(trifluoromethyl)-4H-1,2,4-triazol-3-yl]butanamide (28)

Hydroxybenzotriazole (2.2 mmol) and 1-ethyl-3-(3-dimethylaminopropyl)carbodiimide hydrochloride (2.2 mmol) were added to a stirred solution of 3-(trifluoromethyl)-1H-1,2,4-triazol-5-amine (2.0 mmol) and 4-(2,4-dichlorophenoxy)butanoic acid (2.0 mmol) in dry MeCN (0.15 M) at room temperature. Diisopropylethylamine (5.0 mmol) was added dropwise and the resultant mixture was stirred at room temperature for 18 h. The mixture was concentrated and then partitioned between saturated aqueous NaHCO₃ and CHCl₃, with combined organic extracts being washed with brine, dried over Na₂SO₄ and concentrated. The crude reaction mixture subjected to flash column chromatography (DCM/MeOH) to give 4-(2,4-dichlorophenoxy)-N-[5-(trifluoromethyl)-4H-1,2,4-triazol-3-yl]butanamide (24%) as a beige solid ¹H NMR (600 MHz, CDCl₃) δ 7.35 (t, J=3.5 Hz, 1H), 7.17 (dd, J=8.8, 2.5 Hz, 1H), 6.83 (dd, J=8.8, 3.8 Hz, 1H), 4.12 (t, J=5.9 Hz, 2H), 3.31 (t, J=7.0 Hz, 2H), 2.37-2.27 (m, 2H).

### Example 6: 2-(2,4-dichlorophenoxy)-N-[5-(trifluoromethyl)-4H-1,2,4-triazol-3-yl]propenamide (33)

(COCl)₂ (4.0 mmol) and DMF (1 drop) were added to a stirred solution of 2-(2,4-dichlorophenoxy)propanoic acid (2.0 mmol) in DCM (0.1 M) at room temperature. The resultant mixture was heated to reflux for 2 h. The mixture was cooled and concentrated and MeCN (0.1 M) was added. This solution was added dropwise to a stirred solution of diisopropylethylamine (4.0 mmol) and 3-(trifluoromethyl)-1H-1,2,4-triazol-5-amine (2.0 mmol) in MeCN (0.1 M) at room temperature and was stirred at room temperature for 18 h. The resultant mixture was concentrated and partitioned between saturated aqueous NH₄Cl and CHCl₃, with combined organic extracts being washed with brine, dried over Na₂SO₄ and concentrated. The crude reaction mixture subjected to flash column chromatography (DCM/MeOH) to give 2-(2,4-dichlorophenoxy)-N-[5-(trifluoromethyl)-4H-1,2,4-triazol-3-yl]propenamide (62%) as a white solid ¹H NMR (600 MHz, CDCl₃) δ 12.02 (s, 1H), 9.67 (s, 1H), 7.46 (t, J=11.1 Hz, 1H), 7.25-7.24 (m, 1H), 6.92 (d, J=8.8 Hz, 1H), 4.91 (q, J=6.8 Hz, 1H), 1.70 (d, J=6.8 Hz, 3H).

### Example 7: 2-(2-chloro-4-fluorophenoxy)-N-methyl-N-[1-methyl-3-(trifluoromethyl)-1H-1,2,4-triazol-5-yl]acetamide (42)

K₂CO₃ (1.75 mmol) was added to a stirred solution of 2-(2-chloro-4-fluorophenoxy)-N-[5-(trifluoromethyl)-4H-1,2,4-triazol-3-yl]acetamide (0.7 mmol) in DMF (0.5 M) at room temperature and the resulting mixture was stirred at room temperature for 30 min. Mel (1.75 mmol) was added dropwise and the resultant mixture was stirred at room temperature for 48 h. The resultant mixture was partitioned between saturated aqueous NH₄Cl and EtOAc, with combined organic extracts being washed with brine, dried over Na₂SO₄ and concentrated. The crude reaction mixture subjected to flash column chromatography (hexane/EtOAc) to give 2-(2-chloro-4-fluorophenoxy)-N-methyl-N-[1-methyl-3-(trifluoromethyl)-1H-1,2,4-triazol-5-yl]acetamide (16%) as a white solid ¹H NMR (600 MHz, DMSO-d₆) δ 7.47 (dd, J=8.3, 3.1 Hz, 1H), 7.19 (td, J=8.7, 3.1 Hz, 1H), 7.06 (dd, J=9.2, 4.9 Hz, 1H), 5.24 (s, 2H), 4.06 (s, 3H), 3.36 (s, 3H).

### Example 8: 2-(2-chloro-4-fluorophenoxy)-N-[1-methyl-3-(trifluoromethyl)-1H-1,2,4-triazol-5-yl]acetamide (41)

K₂CO₃ (2.2 mmol) was added to a stirred solution of 2-(2-chloro-4-fluorophenoxy)-N-[5-(trifluoromethyl)-4H-1,2,4-triazol-3-yl]acetamide (1.0 mmol) in DMF (0.5 M) at room temperature and the resulting mixture was stirred at room temperature for 30 min. Mel (1.2 mmol) was added dropwise and the resultant mixture was stirred at room temperature for 24 h. The resultant mixture was partitioned between water and EtOAc, with combined organic extracts being washed with brine, dried over Na₂SO₄ and concentrated. The crude reaction mixture subjected to flash column chromatography (hexane/EtOAc) to give 2-(2-chloro-4-fluorophenoxy)-N-[1-methyl-3-(trifluoromethyl)-1H-1,2,4-triazol-5-yl]acetamide (54%) as a white solid ¹H NMR (600 MHz, DMSO-d₆) δ 11.15 (s, 1H), 7.59-7.38 (m, 1H), 7.30-7.07 (m, 2H), 4.99 (s, 2H), 3.78 (s, 3H).

LC-MS measurements were performed on an Agilent 1100 Series LC/MSD system with DAD\LSD Alltech 2000ES and Agilent LC\MSD VL (G1956B), SL (G1956B) mass-spectrometer. Agilent 1200 Series LC/MSD system with DAD\LSD Alltech 3300 and Agilent LC\SD G6130A, G6120B mass-spectrometer. Agilent Technologies 1260 Infinity LC/MSD system with DAD\LSD Alltech 3300 and Agilent LC\SD G6120B mass-spectrometer. Agilent Technologies 1260 Infinity II LC/MSD system with DAD\LSD G7102A 1290 Infinity II and Agilent LC\SD G6120B mass-spectrometer. All the LC/MS data were obtained using positive/negative mode switching. LCMS Acquisition parameters: Column Agilent Poroshell 120 SB-C18 4.6×30 mm 2.7 μm with UHPLC Guard Infinity Lab Poroshell 120 SB-C18 4.6×5 mm 2.7 μm Temperature 60 C Mobile phase A—acetonitrile:water (99:1%), 0.1% formic acid B—water (0.1% formic acid) Flow rate 3 ml/min Gradient: 0.01 min −99% B, 1.5 min −0% B, 2.2 min 0% B, 2.21 min 99% B.

HPLC purification was performed using Agilent 1260 Infinity systems equipped with DAD and mass-detector. Waters Sunfire C18 OBD Prep Column, 100 A, 5 μm, 19 mm×100 mm with SunFire C18 Prep Guard Cartridge, 100 A, 10 μm, 19 mm×10 mm was used. In some cases, ammonia or trifluoroacetic acid (TFA) was used as an additive to improve the separation of the products. In these cases, free bases and TFA salts of the products were formed, respectively.

Examples of herbicidal compounds of the present invention are presented in Table 1.

TABLE 1
Example herbicidal compounds

| Compound | Structure | NMR or HPLC Data |
|---|---|---|
| 1 | 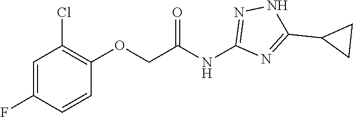 [SMILES: O=C(COc1ccc(F)cc1Cl)Nc1n[nH]c(C2CC2)n1] | 1.162 min |
| 2 | 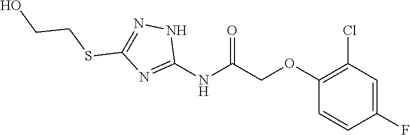 [SMILES: O=C(COc1ccc(F)cc1Cl)Nc1nc(SCCO)n[nH]1] | 0.942 min |
| 3 | 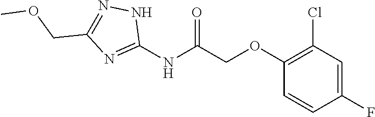 [SMILES: COCc1n[nH]c(NC(=O)COc2ccc(F)cc2Cl)n1] | 1.022 min |
| 4 | 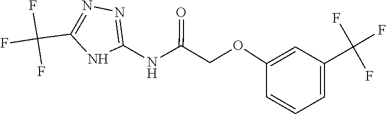 [SMILES: O=C(COc1cccc(C(F)(F)F)c1)Nc1nnc(C(F)(F)F)[nH]1] | 1.179 min |
| 5 | 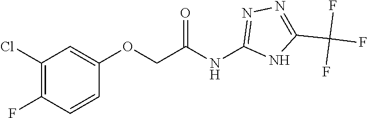 [SMILES: O=C(COc1ccc(F)c(Cl)c1)Nc1nnc(C(F)(F)F)[nH]1] | 1.304 min |
| 6 | 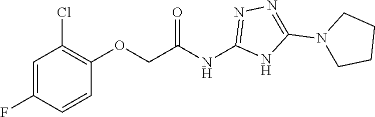 [SMILES: O=C(COc1ccc(F)cc1Cl)Nc1nnc(N2CCCC2)[nH]1] | 1.454 min |
| 7 | 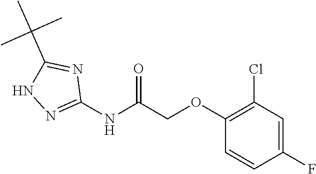 [SMILES: CC(C)(C)c1nc(NC(=O)COc2ccc(F)cc2Cl)n[nH]1] | 1.421 min |
| 8 | 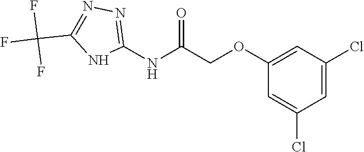 [SMILES: O=C(COc1cc(Cl)cc(Cl)c1)Nc1nnc(C(F)(F)F)[nH]1] | 1.388 min |
| 9 | 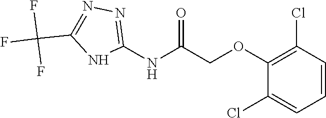 [SMILES: O=C(COc1c(Cl)cccc1Cl)Nc1nnc(C(F)(F)F)[nH]1] | 1.169 min |
| 10 | 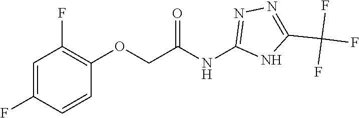 [SMILES: O=C(COc1ccc(F)cc1F)Nc1nnc(C(F)(F)F)[nH]1] | 1.297 min |
| 11 | 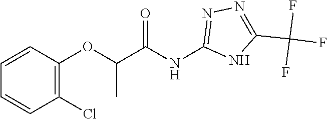 [SMILES: CC(Oc1ccccc1Cl)C(=O)Nc1nnc(C(F)(F)F)[nH]1] | 1.402 min |
| 12 | 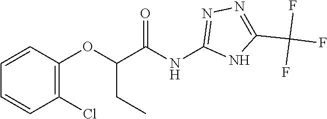 [SMILES: CCC(Oc1ccccc1Cl)C(=O)Nc1nnc(C(F)(F)F)[nH]1] | 1.288 min |
| 13 | 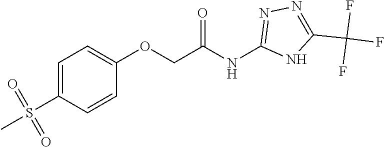 [SMILES: CS(=O)(=O)c1ccc(OCC(=O)Nc2nnc(C(F)(F)F)[nH]2)cc1] | 1.094 min |
| 14 | 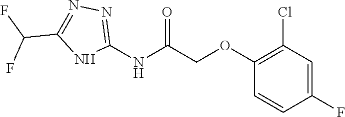 [SMILES: O=C(COc1ccc(F)cc1Cl)Nc1nnc(C(F)F)[nH]1] | 1.228 min |
| 15 | 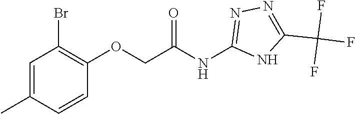 [SMILES: Cc1ccc(OCC(=O)Nc2nnc(C(F)(F)F)[nH]2)c(Br)c1] | 1.430 min |
| 16 | 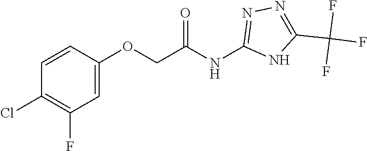 [SMILES: O=C(COc1ccc(Cl)c(F)c1)Nc1nnc(C(F)(F)F)[nH]1] | 1.311 min |
| 17 | 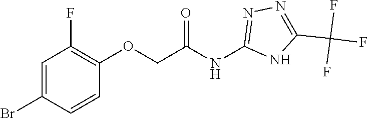 [SMILES: O=C(COc1ccc(Br)cc1F)Nc1nnc(C(F)(F)F)[nH]1] | 1.155 min |
| 18 | 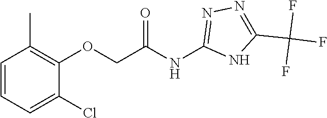 [SMILES: Cc1cccc(Cl)c1OCC(=O)Nc1nnc(C(F)(F)F)[nH]1] | 1.173 min |
| 19 | 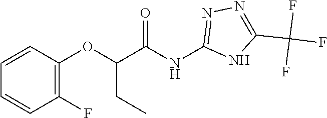 [SMILES: CCC(Oc1ccccc1F)C(=O)Nc1nnc(C(F)(F)F)[nH]1] | 1.166 min |
| 20 | 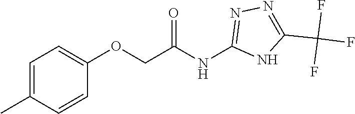 [SMILES: Cc1ccc(OCC(=O)Nc2nnc(C(F)(F)F)[nH]2)cc1] | 1.163 min |
| 21 | 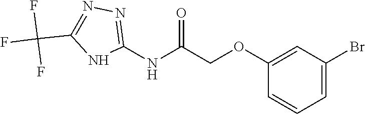 [SMILES: O=C(COc1cccc(Br)c1)Nc1nnc(C(F)(F)F)[nH]1] | 1.307 min |
| 22 | 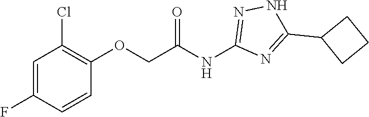 [SMILES: O=C(COc1ccc(F)cc1Cl)Nc1n[nH]c(C2CCC2)n1] | 1.070 min |
| 23 | 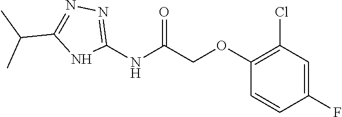 [SMILES: CC(C)c1nnc(NC(=O)COc2ccc(F)cc2Cl)[nH]1] | 1.217 min |
| 24 | 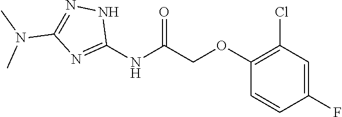 [SMILES: CN(C)c1n[nH]c(NC(=O)COc2ccc(F)cc2Cl)n1] | 1.384 min |
| 25 | 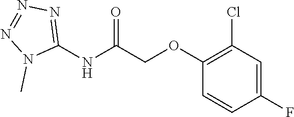 [SMILES: Cn1nnnc1NC(=O)COc1ccc(F)cc1Cl] | ¹H NMR (600 MHz, DMSO, 80° C.) δ 7.29 (d, J = 5.4 Hz, 1H), 7.14-6.92 (m, 2H), 4.63 (s, 2H), 3.65 (s, 3H). |
| 26 | 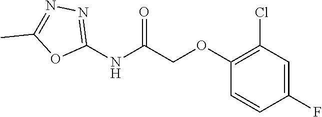 [SMILES: Cc1nnc(NC(=O)COc2ccc(F)cc2Cl)o1] | ¹H NMR (600 MHz, DMSO, 80° C.) δ 7.30 (d, J = 7.3 Hz, 1H), 7.05 (bs, 2H), 4.66 (s, 2H), 2.32 (bs, 3H). |
| 27 | 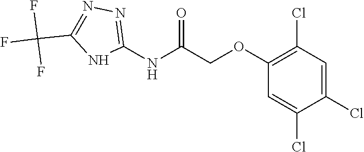 [SMILES: O=C(COc1cc(Cl)c(Cl)cc1Cl)Nc1nnc(C(F)(F)F)[nH]1] | ¹H NMR (600 MHZ, CDCl₃) δ 7.35 (t, J = 3.5 Hz, 1H), 7.17 (dd, J = 8.8, 2.5 Hz, 1H), 6.83 (dd, J = 8.8, 3.8 Hz, 1H), 4.12 (t, J = 5.9 Hz, 2H), 3.31 (t, J = 7.0 Hz, 2H), 2.37- 2.27 (m, 2H). |
| 28 | 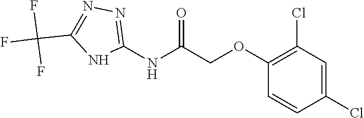 [SMILES: O=C(COc1ccc(Cl)cc1Cl)Nc1nnc(C(F)(F)F)[nH]1] | ¹H NMR (600 MHz, CDCl₃) δ 7.35 (t, J = 3.5 Hz, 1H), 7.17 (dd, J = 8.8, 2.5 Hz, 1H), 6.83 (dd, J = 8.8, 3.8 Hz, 1H), 4.12 (t, J = 5.9 Hz, 2H), 3.31 (t, J = 7.0 Hz, 2H), 2.37- 2.27 (m, 2H). |
| 29 | 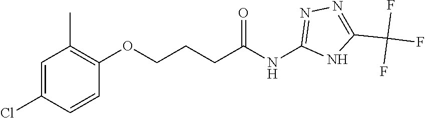 [SMILES: Cc1cc(Cl)ccc1OCCCC(=O)Nc1nnc(C(F)(F)F)[nH]1] | ¹H NMR (600 MHz, DMSO) δ 7.95 (bs, 2H), 7.19 (d, J = 2.2 Hz, 1H), 7.17 (dd, J = 8.7, 2.5 Hz, 1H), 6.92 (d, J = 8.7 Hz, 1H), 4.05 (t, J = 6.2 Hz, 2H), 3.16 (t, J = 7.1 Hz, 2H), 2.12 (dd, J = 13.3, 6.7 Hz, 2H), 2.09 (s, 3H). |
| 30 | 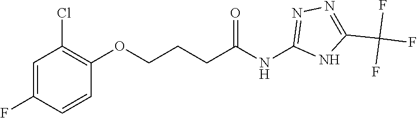 [SMILES: O=C(CCCOc1ccc(F)cc1Cl)Nc1nnc(C(F)(F)F)[nH]1] | ¹H NMR (600 MHz, DMSO) δ 7.94 (bs, 2 H), 7.42-7.39 (m, 1H), 7.16 (dd, J = 7.4, 1.9 Hz, 2H), 4.12 (t, J = 6.3 Hz, 2H), 3.16 (t, J = 7.1 Hz, 2H), 2.12 (q, J = 6.7 Hz, 2H). |
| 31 | 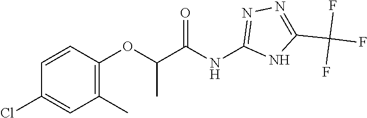 [SMILES: Cc1cc(Cl)ccc1OC(C)C(=O)Nc1nnc(C(F)(F)F)[nH]1] | ¹H NMR (600 MHz, DMSO) δ 14.36 (s, 1H), 12.23 (s, 1H), 7.25 (d, J = 2.1 Hz, 1H), 7.17 (dd, J = 8.7, 2.4 Hz, 1H), 6.80 (d, J = 8.8 Hz, 1H), 5.02 (q, J = 6.6 Hz, 1H), 2.23 (s, 3H), 1.58 (d, J = 6.6 Hz, 3H). |
| 32 | 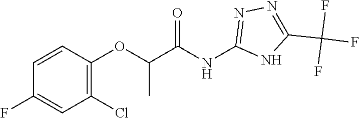 [SMILES: CC(Oc1ccc(F)cc1Cl)C(=O)Nc1nnc(C(F)(F)F)[nH]1] | ¹H NMR (600 MHz, DMSO) δ 14.36 (s, 1H), 12.26 (s, 1H), 7.47 (dd, J = 8.3, 3.1 Hz, 1H), 7.19-7.13 (m, 1H), 7.05 (dd, J = 9.2, 4.9 Hz, 1H), 5.06 (q, J = 6.6 Hz, 1H), 1.60 (d, J = 6.6 Hz, 3H). |
| 33 | 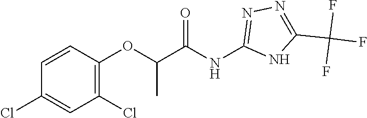 [SMILES: CC(Oc1ccc(Cl)cc1Cl)C(=O)Nc1nnc(C(F)(F)F)[nH]1] | ¹H NMR (600 MHz, CDCl₃) δ 12.02 (s, 1H), 9.67 (s, 1H), 7.46 (t, J = 11.1 Hz, 1H), 7.25- 7.24 (m, 1H), 6.92 (d, J = 8.8 Hz, 1H), 4.91 (q, J = 6.8 Hz, 1H), 1.70 (d, J = 6.8 Hz, 3H). |
| 34 | 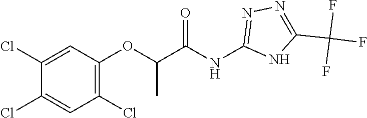 [SMILES: CC(Oc1cc(Cl)c(Cl)cc1Cl)C(=O)Nc1nnc(C(F)(F)F)[nH]1] | ¹H NMR (600 MHz, CDCl₃) δ 12.40 (s, 1H), 9.85 (s, 1H), 7.55 (s, 1H), 8.43-5.98 (m, 3H), 7.09 (s, 1H), 4.93 (q, J = 6.8 Hz, 1H), 1.73 (d, J = 6.8 Hz, 3H) |
| 35 | 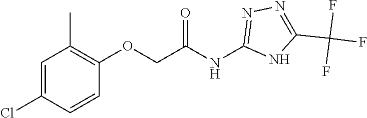 [SMILES: Cc1cc(Cl)ccc1OCC(=O)Nc1nnc(C(F)(F)F)[nH]1] | ¹H NMR (600 MHz, DMSO) δ 8.03 (s, 2H), 7.25 (d, J = 2.2 Hz, 1H), 7.13 (dd, J = 8.7, 2.5 Hz, 1H), 7.05 (d, J = 8.8 Hz, 1H), 5.42 (s, 2H), 2.23 (s, 3H). |
| 36 | 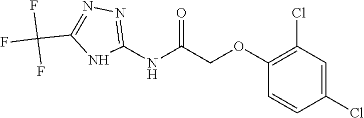 [SMILES: O=C(COc1ccc(Cl)cc1Cl)Nc1nnc(C(F)(F)F)[nH]1] | 1.372 min |
| 37 | 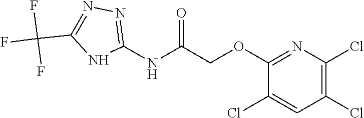 [SMILES: O=C(COc1nc(Cl)c(Cl)cc1Cl)Nc1nnc(C(F)(F)F)[nH]1] | ¹H NMR (600 MHz, DMSO) δ 14.31 (s, 1H), 12.27 (s, J = 85.9 Hz, 1H), 8.46 (s, 1H), 5.16 (s, 1H). |
| 38 | 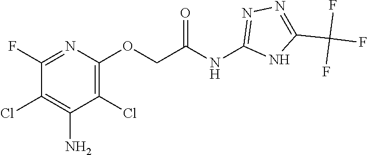 [SMILES: Nc1c(Cl)c(F)nc(OCC(=O)Nc2nnc(C(F)(F)F)[nH]2)c1Cl] | ¹H NMR (600 MHz, DMSO) δ 14.27 (s, 1H), 12.18 (s, 1H), 7.05 (s, 2H), 5.02 (s, 2H). |
| 39 | 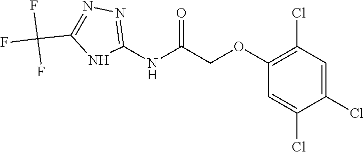 [SMILES: O=C(COc1cc(Cl)c(Cl)cc1Cl)Nc1nnc(C(F)(F)F)[nH]1] | ¹H NMR (600 MHz, CDCl₃) δ 12.04 (s, 1H), 9.56 (s, 1H), 7.57 (s, 1H), 7.07 (s, 1H), 4.77 (s, 2H). |
| 40 | 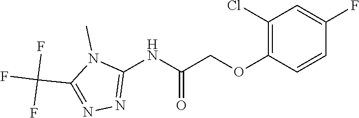 [SMILES: Cn1c(NC(=O)COc2ccc(F)cc2Cl)nnc1C(F)(F)F] | ¹H NMR (600 MHz, DMSO) δ 12.64 (s, 1H), 7.44 (dd, J = 8.3, 3.1 Hz, 1H), 7.19- 7.13 (m, 1H), 7.07 (dd, J = 9.2, 4.9 Hz, 1H), 4.79 (s, 2H), 3.25 (s, 3H). |
| 41 | 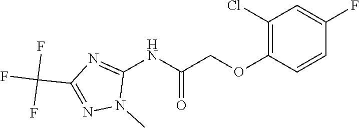 [SMILES: Cn1nc(C(F)(F)F)nc1NC(=O)COc1ccc(F)cc1Cl] | ¹H NMR (600 MHz, DMSO) δ 11.15 (s, 1H), 7.59-7.38 (m, 1H), 7.30-7.07 (m, 2H), 4.99 (s, 2H), 3.78 (s, 3H). |
| 42 | 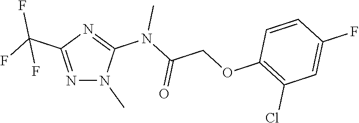 [SMILES: CN(C(=O)COc1ccc(F)cc1Cl)c1nc(C(F)(F)F)nn1C] | ¹H NMR (600 MHz, DMSO) δ 7.47 (dd, J = 8.3, 3.1 Hz, 1H), 7.19 (td, J = 8.7, 3.1 Hz, 1H), 7.06 (dd, J = 9.2, 4.9 Hz, 1H), 5.24 (s, 2H), 4.06 (s, 3H), 3.36 (s, 3H). |
| 43 | 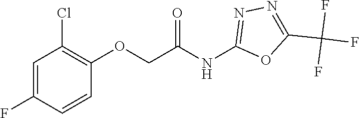 [SMILES: O=C(COc1ccc(F)cc1Cl)Nc1nnc(C(F)(F)F)o1] | ¹H NMR (600 MHz, DMSO) δ 7.38 (dd, J = 8.0, 2.4 Hz, 1H), 7.14-7.08 (m, 1H), 6.94 (dd, J = 8.8, 4.7 Hz, 1H), 4.59 (s, 2H). |
| 44 | 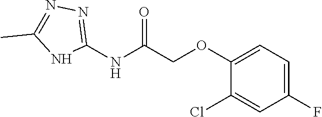 [SMILES: Cc1nnc(NC(=O)COc2ccc(F)cc2Cl)[nH]1] | ¹H NMR (600 MHz, CDCl₃) δ 7.18 (d, J = 7.4 Hz, 1H), 6.93 (dd, J = 11.3, 6.2 Hz, 2H), 6.16 (bs, 2H), 5.31 (s, 2H), 2.25 (s, 3H). |
| 45 | 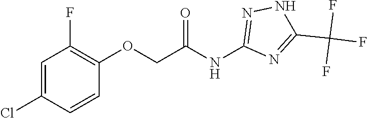 [SMILES: O=C(COc1ccc(Cl)cc1F)Nc1n[nH]c(C(F)(F)F)n1] | ¹H NMR (600 MHz, CDCl₃) δ 12.04 (s, 1H), 9.59 (s, 1H), 7.21 (dd, J = 10.6, 2.4 Hz, 1H), 7.14-7.11 (m, 1H), 6.96 (t, J = 8.7 Hz, 1H), 4.77 (s, 2H). |
| 46 | 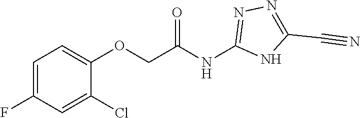 [SMILES: N#Cc1nnc(NC(=O)COc2ccc(F)cc2Cl)[nH]1] | ¹H NMR (600 MHz, DMSO) δ 14.63 (s, 1H), 12.22 (s, 1H), 7.46 (dd, J = 8.3, 2.8 Hz, 1H), 7.18-7.11 (m, 2H), 4.98 (s, 2H). |
| 47 | 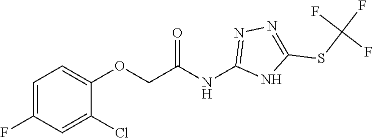 [SMILES: O=C(COc1ccc(F)cc1Cl)Nc1nnc(SC(F)(F)F)[nH]1] | ¹H NMR (600 MHz, DMSO) δ 14.29 (s, 1H), 12.14 (s, 1H), 7.46 (dd, J = 8.3, 2.9 Hz, 1H), 7.19-7.12 (m, 2H), 4.97 (s, 2H). |
| 48 | 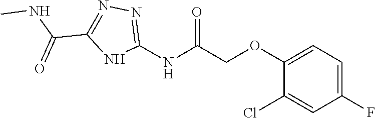 [SMILES: CNC(=O)c1nnc(NC(=O)COc2ccc(F)cc2Cl)[nH]1] | ¹H NMR (600 MHz, DMSO) δ 13.88 (s, 1H), 11.88 (s, 1H), 8.22 (s, 1H), 7.46 (dd, J = 8.3, 2.8 Hz, 1H), 7.21-7.08 (m, 2H), 4.97 (s, 2H), 2.75 (s, 3H). |

Further examples of compounds according to the invention are discussed below in Table 2.

TABLE 2
Further exemplary compounds of the present invention

| No. | Structure |
|---|---|
| 49 | 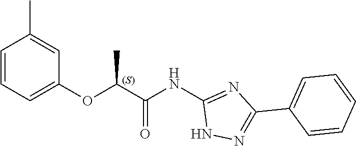 |
| 50 | 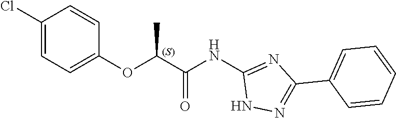 |
| 51 | 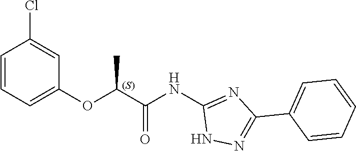 |
| 52 | 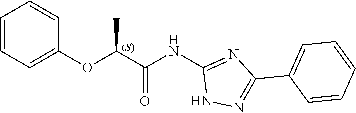 |
| 53 | 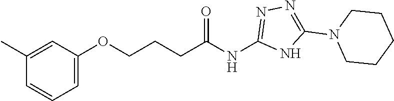 [SMILES: Cc1cccc(OCCCC(=O)Nc2nnc(N3CCCCC3)[nH]2)c1] |
| 54 | 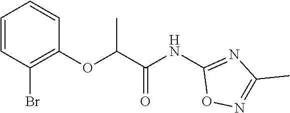 [SMILES: Cc1noc(NC(=O)C(C)Oc2ccccc2Br)n1] |
| 55 | 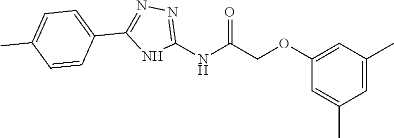 |
| 56 | 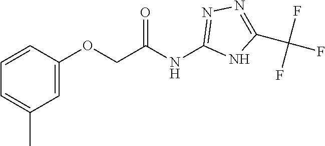 [SMILES: Cc1cccc(OCC(=O)Nc2nnc(C(F)(F)F)[nH]2)c1] |
| 57 | 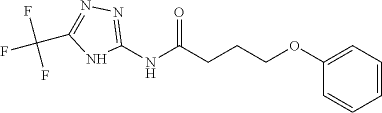 [SMILES: O=C(CCCOc1ccccc1)Nc1nnc(C(F)(F)F)[nH]1] |
| 58 | 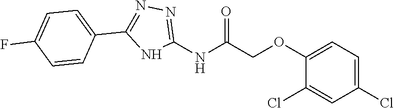 [SMILES: O=C(COc1ccc(Cl)cc1Cl)Nc1nnc(-c2ccc(F)cc2)[nH]1] |
| 59 | 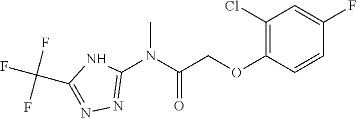 [SMILES: CN(C(=O)COc1ccc(F)cc1Cl)c1nnc(C(F)(F)F)[nH]1] |
| 60 | 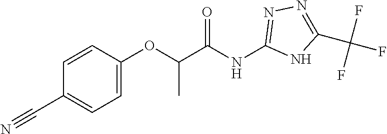 [SMILES: CC(Oc1ccc(C#N)cc1)C(=O)Nc1nnc(C(F)(F)F)[nH]1] |
| 61 | 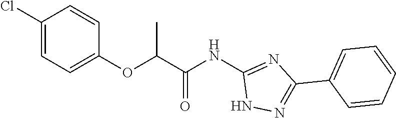 [SMILES: CC(Oc1ccc(Cl)cc1)C(=O)Nc1nc(-c2ccccc2)n[nH]1] |
| 62 | 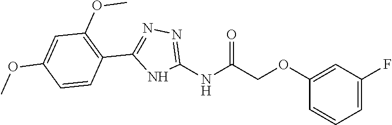 [SMILES: COc1ccc(-c2nnc(NC(=O)COc3cccc(F)c3)[nH]2)c(OC)c1] |
| 63 | 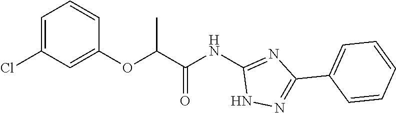 [SMILES: CC(Oc1cccc(Cl)c1)C(=O)Nc1nc(-c2ccccc2)n[nH]1] |
| 64 | 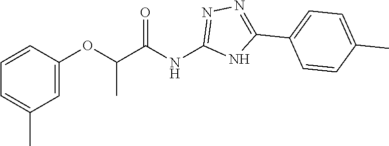 [SMILES: Cc1ccc(-c2nnc(NC(=O)C(C)Oc3cccc(C)c3)[nH]2)cc1] |
| 65 | 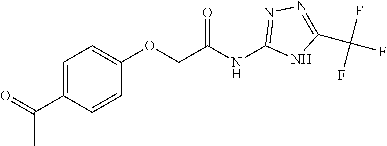 [SMILES: CC(=O)c1ccc(OCC(=O)Nc2nnc(C(F)(F)F)[nH]2)cc1] |
| 66 | 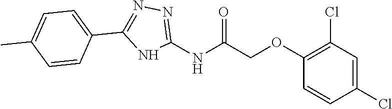 [SMILES: Cc1ccc(-c2nnc(NC(=O)COc3ccc(Cl)cc3Cl)[nH]2)cc1] |
| 67 | 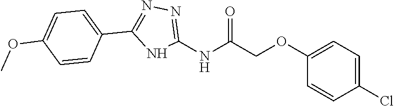 [SMILES: COc1ccc(-c2nnc(NC(=O)COc3ccc(Cl)cc3)[nH]2)cc1] |
| 68 | 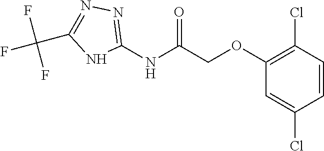 [SMILES: O=C(COc1cc(Cl)ccc1Cl)Nc1nnc(C(F)(F)F)[nH]1] |
| 69 | 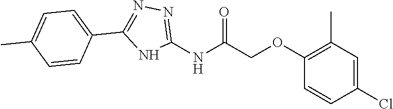 [SMILES: Cc1ccc(-c2nnc(NC(=O)COc3ccc(Cl)cc3C)[nH]2)cc1] |
| 70 | 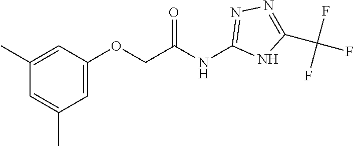 [SMILES: Cc1cc(C)cc(OCC(=O)Nc2nnc(C(F)(F)F)[nH]2)c1] |
| 71 | 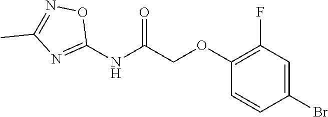 [SMILES: Cc1noc(NC(=O)COc2ccc(Br)cc2F)n1] |
| 72 | 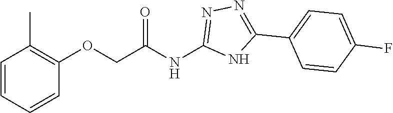 [SMILES: Cc1ccccc1OCC(=O)Nc1nnc(-c2ccc(F)cc2)[nH]1] |
| 73 | 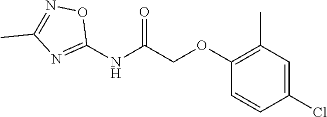 [SMILES: Cc1noc(NC(=O)COc2ccc(Cl)cc2C)n1] |
| 74 | 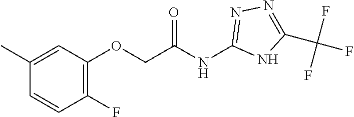 [SMILES: Cc1ccc(F)c(OCC(=O)Nc2nnc(C(F)(F)F)[nH]2)c1] |
| 75 | 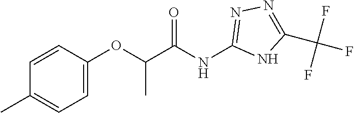 [SMILES: Cc1ccc(OC(C)C(=O)Nc2nnc(C(F)(F)F)[nH]2)cc1] |
| 76 | 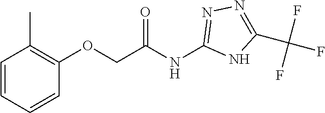 [SMILES: Cc1ccccc1OCC(=O)Nc1nnc(C(F)(F)F)[nH]1] |
| 77 | 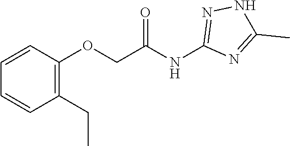 [SMILES: CCc1ccccc1OCC(=O)Nc1n[nH]c(C)n1] |
| 78 | 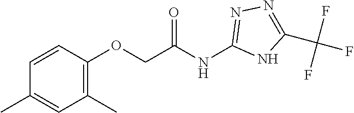 [SMILES: Cc1ccc(OCC(=O)Nc2nnc(C(F)(F)F)[nH]2)c(C)c1] |
| 79 | 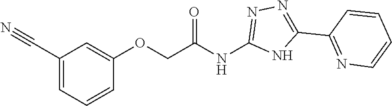 [SMILES: N#Cc1cccc(OCC(=O)Nc2nnc(-c3ccccn3)[nH]2)c1] |
| 80 | 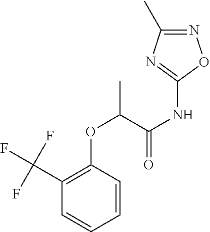 [SMILES: Cc1noc(NC(=O)C(C)Oc2ccccc2C(F)(F)F)n1] |
| 81 | 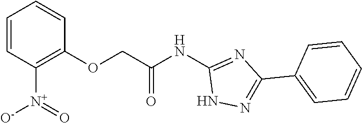 [SMILES: O=C(COc1ccccc1[N+](=O)[O-])Nc1nc(-c2ccccc2)n[nH]1] |
| 82 | 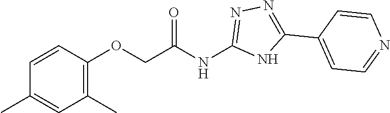 [SMILES: Cc1ccc(OCC(=O)Nc2nnc(-c3ccncc3)[nH]2)c(C)c1] |
| 83 | 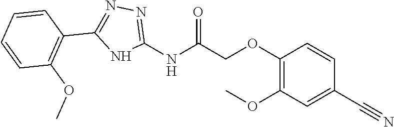 [SMILES: COc1cc(C#N)ccc1OCC(=O)Nc1nnc(-c2ccccc2OC)[nH]1] |
| 84 | 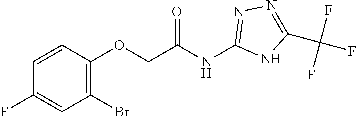 [SMILES: O=C(COc1ccc(F)cc1Br)Nc1nnc(C(F)(F)F)[nH]1] |
| 85 | 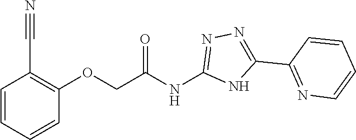 [SMILES: N#Cc1ccccc1OCC(=O)Nc1nnc(-c2ccccn2)[nH]1] |
| 86 | 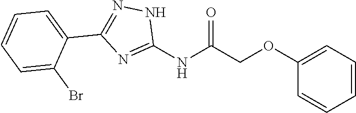 [SMILES: O=C(COc1ccccc1)Nc1nc(-c2ccccc2Br)n[nH]1] |
| 87 | 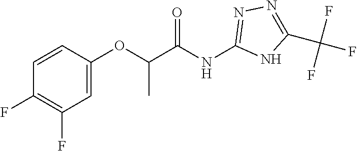 [SMILES: CC(Oc1ccc(F)c(F)c1)C(=O)Nc1nnc(C(F)(F)F)[nH]1] |
| 106 | 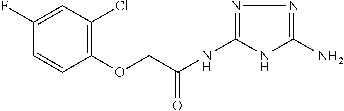 [SMILES: Nc1nnc(NC(=O)COc2ccc(F)cc2Cl)[nH]1] |
| 107 | 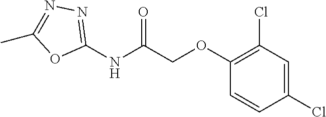 [SMILES: Cc1nnc(NC(=O)COc2ccc(Cl)cc2Cl)o1] |
| 108 | 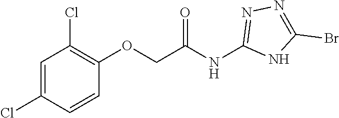 [SMILES: O=C(COc1ccc(Cl)cc1Cl)Nc1nnc(Br)[nH]1] |
| 109 | 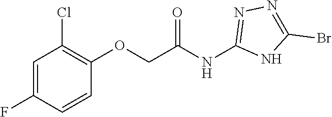 [SMILES: O=C(COc1ccc(F)cc1Cl)Nc1nnc(Br)[nH]1] |
| 110 | 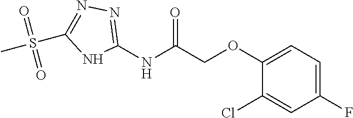 [SMILES: CS(=O)(=O)c1nnc(NC(=O)COc2ccc(F)cc2Cl)[nH]1] |

### Biological Examples

Seeds of a variety of test species are grown in a sandy loam soil mixture, Lolium multiflorum (LOLMU), Amaranthus retroflexus (AMARE), Echinochloa crus-galli (ECHCG), Veronica persica (VERPE).

After sowing, at growth stage BBCH 12-14 (post-emergence), the plants are sprayed with an aqueous spray solution derived from the formulation of the technical active ingredient in acetone/water (50:50) solution containing 0.5% Tween 20 (polyoxyethlyene sorbitan monolaureate, CAS RN 9005-64-5). The test compounds are applied at the required concentration of active ingredient in g/ha. The test plants are then grown in a glasshouse under controlled environmental conditions and watered regularly as required. After 14±1 days post application (for post-emergence test), the test is evaluated by assessing the percentage damage caused to the plants in comparison with the untreated plots. The biological activities for post-emergence testing are shown below (Table 3) as a % visual injury.

TABLE 3
Visual injury caused to plants from post-emergence testing from a range of
compounds

| | | | LOLMU | ECHCG | VERPE | AMARE |
|---|---|---|---|---|---|---|
| | | | % | % | % | % |
| Compound | | Rate | visual | visual | visual | visual |
| number | Structure | g/ha | Injury | Injury | Injury | Injury |
| 88 | 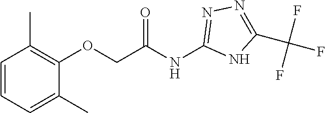 [SMILES: Cc1cccc(C)c1OCC(=O)Nc1nnc(C(F)(F)F)[nH]1] | 500 | 0 | 0 | 20 | 0 |
| 89 | 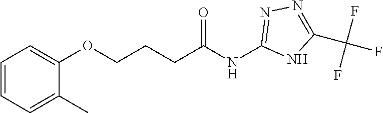 [SMILES: Cc1ccccc1OCCCC(=O)Nc1nnc(C(F)(F)F)[nH]1] | 500 | 0 | 0 | 95 | 40 |
| 90 | 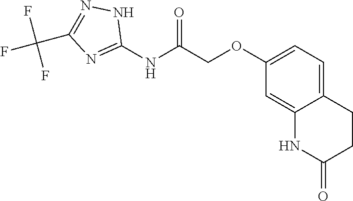 [SMILES: O=C(COc1ccc2c(c1)NC(=O)CC2)Nc1nc(C(F)(F)F)n[nH]1] | 500 | 0 | 0 | 70 | 0 |
| 91 | 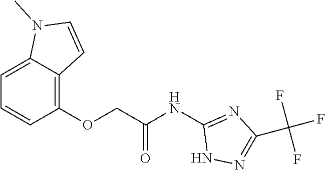 [SMILES: Cn1ccc2c(OCC(=O)Nc3nc(C(F)(F)F)n[nH]3)cccc21] | 500 | 0 | 0 | 70 | 0 |
| 92 | 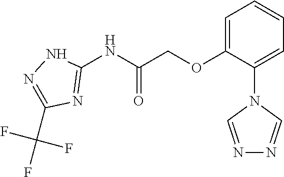 [SMILES: O=C(COc1ccccc1-n1cnnc1)Nc1nc(C(F)(F)F)n[nH]1] | 500 | 0 | 0 | 70 | 0 |
| 7 | 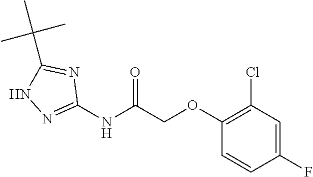 [SMILES: CC(C)(C)c1nc(NC(=O)COc2ccc(F)cc2Cl)n[nH]1] | 500 | 40 | 80 | 60 | 60 |
| 10 | 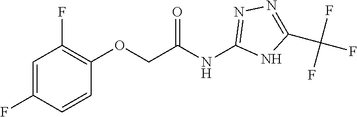 [SMILES: O=C(COc1ccc(F)cc1F)Nc1nnc(C(F)(F)F)[nH]1] | 500 | 0 | 30 | 80 | 40 |
| 17 | 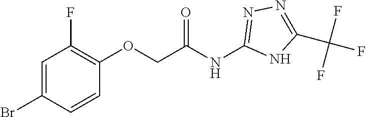 [SMILES: O=C(COc1ccc(Br)cc1F)Nc1nnc(C(F)(F)F)[nH]1] | 500 | 0 | 0 | 70 | 60 |
| 1 | 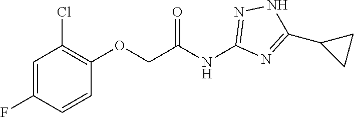 [SMILES: O=C(COc1ccc(F)cc1Cl)Nc1n[nH]c(C2CC2)n1] | 500 | 40 | 90 | 60 | 100 |
| 23 | 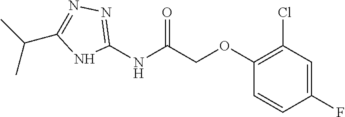 [SMILES: CC(C)c1nnc(NC(=O)COc2ccc(F)cc2Cl)[nH]1] | 500 | 40 | 90 | 100 | 100 |
| 22 | 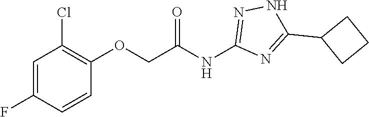 [SMILES: O=C(COc1ccc(F)cc1Cl)Nc1n[nH]c(C2CCC2)n1] | 500 | 40 | 90 | 90 | 100 |
| 16 | 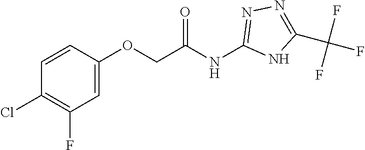 [SMILES: O=C(COc1ccc(Cl)c(F)c1)Nc1nnc(C(F)(F)F)[nH]1] | 500 | 0 | 20 | 70 | 100 |
| 24 | 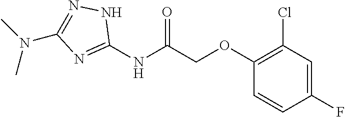 [SMILES: CN(C)c1n[nH]c(NC(=O)COc2ccc(F)cc2Cl)n1] | 500 | 0 | 70 | 50 | 70 |
| 15 | 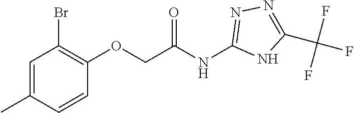 [SMILES: Cc1ccc(OCC(=O)Nc2nnc(C(F)(F)F)[nH]2)c(Br)c1] | 500 | 0 | 0 | 50 | 0 |
| 18 | 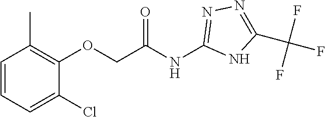 [SMILES: Cc1cccc(Cl)c1OCC(=O)Nc1nnc(C(F)(F)F)[nH]1] | 500 | 0 | 0 | 70 | 0 |
| 4 | 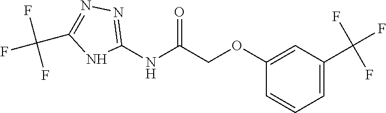 [SMILES: O=C(COc1cccc(C(F)(F)F)c1)Nc1nnc(C(F)(F)F)[nH]1] | 500 | 0 | 20 | 80 | 99 |
| 5 | 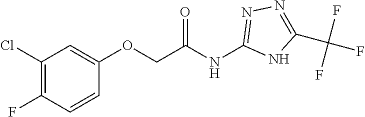 [SMILES: O=C(COc1ccc(F)c(Cl)c1)Nc1nnc(C(F)(F)F)[nH]1] | 500 | 0 | 30 | 98 | 80 |
| 6 | 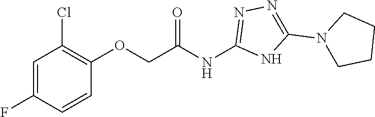 [SMILES: O=C(COc1ccc(F)cc1Cl)Nc1nnc(N2CCCC2)[nH]1] | 500 | 0 | 20 | 50 | 50 |
| 21 | 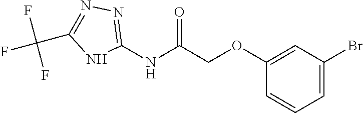 [SMILES: O=C(COc1cccc(Br)c1)Nc1nnc(C(F)(F)F)[nH]1] | 500 | 20 | 0 | 60 | 0 |
| 14 | 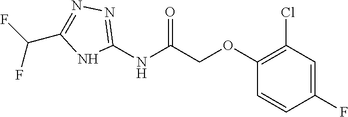 [SMILES: O=C(COc1ccc(F)cc1Cl)Nc1nnc(C(F)F)[nH]1] | 500 | 60 | 90 | 60 | 100 |
| 3 | 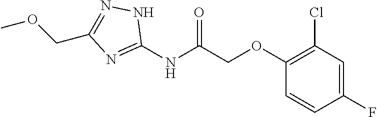 [SMILES: COCc1n[nH]c(NC(=O)COc2ccc(F)cc2Cl)n1] | 500 | 60 | 90 | 90 | 100 |
| 93 | 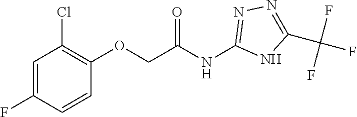 [SMILES: O=C(COc1ccc(F)cc1Cl)Nc1nnc(C(F)(F)F)[nH]1] | 500 | 70 | 80 | 90 | 100 |
| 94 | 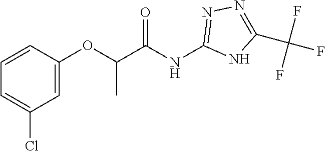 [SMILES: CC(Oc1cccc(Cl)c1)C(=O)Nc1nnc(C(F)(F)F)[nH]1] | 1000 | 0 | 10 | 50 | 50 |
| 26 | 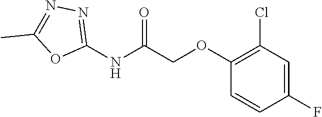 [SMILES: Cc1nnc(NC(=O)COc2ccc(F)cc2Cl)o1] | 500 | 50 | 90 | 95 | 90 |
| 25 | 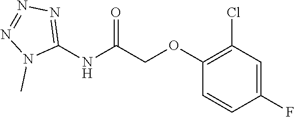 [SMILES: Cn1nnnc1NC(=O)COc1ccc(F)cc1Cl] | 500 | 70 | 0 | 70 | 40 |
| 28 | 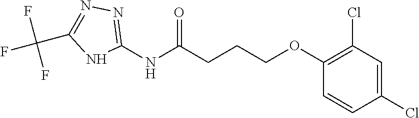 [SMILES: O=C(CCCOc1ccc(Cl)cc1Cl)Nc1nnc(C(F)(F)F)[nH]1] | 500 | 0 | 10 | 60 | 70 |
| 29 | 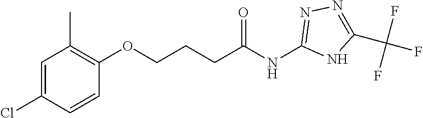 [SMILES: Cc1cc(Cl)ccc1OCCCC(=O)Nc1nnc(C(F)(F)F)[nH]1] | 500 | 60 | 0 | 80 | 60 |
| 30 | 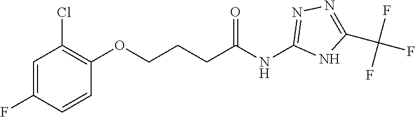 [SMILES: O=C(CCCOc1ccc(F)cc1Cl)Nc1nnc(C(F)(F)F)[nH]1] | 500 | 70 | 30 | 60 | 80 |
| 35 | 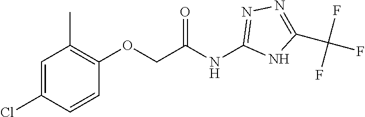 [SMILES: Cc1cc(Cl)ccc1OCC(=O)Nc1nnc(C(F)(F)F)[nH]1] | 1000 | 20 | 30 | 60 | 80 |
| 36 | 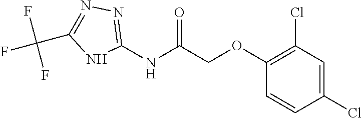 [SMILES: O=C(COc1ccc(Cl)cc1Cl)Nc1nnc(C(F)(F)F)[nH]1] | 1000 | 40 | 30 | 98 | 99 |
| 95 | 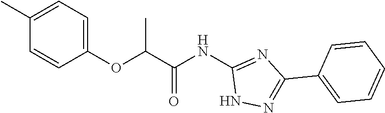 [SMILES: Cc1ccc(OC(C)C(=O)Nc2nc(-c3ccccc3)n[nH]2)cc1] | 500 | 0 | 0 | 0 | 50 |
| 9 | 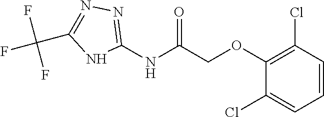 [SMILES: O=C(COc1c(Cl)cccc1Cl)Nc1nnc(C(F)(F)F)[nH]1] | 500 | 0 | 0 | 0 | 20 |
| 12 | 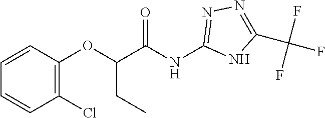 [SMILES: CCC(Oc1ccccc1Cl)C(=O)Nc1nnc(C(F)(F)F)[nH]1] | 500 | 0 | 10 | 60 | 70 |
| 13 | 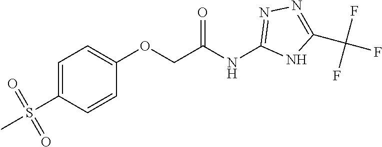 [SMILES: CS(=O)(=O)c1ccc(OCC(=O)Nc2nnc(C(F)(F)F)[nH]2)cc1] | 500 | 60 | 0 | 80 | 60 |
| 19 | 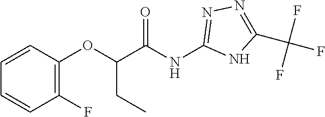 [SMILES: CCC(Oc1ccccc1F)C(=O)Nc1nnc(C(F)(F)F)[nH]1] | 500 | 0 | 0 | 20 | 0 |
| 20 | 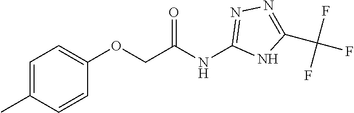 [SMILES: Cc1ccc(OCC(=O)Nc2nnc(C(F)(F)F)[nH]2)cc1] | 500 | 0 | 30 | 40 | 0 |
| 42 | 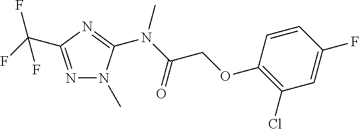 [SMILES: CN(C(=O)COc1ccc(F)cc1Cl)c1nc(C(F)(F)F)nn1C] | 500 | 40 | 60 | 60 | 90 |
| 96 | 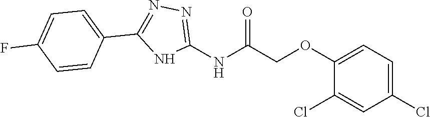 [SMILES: O=C(COc1ccc(Cl)cc1Cl)Nc1nnc(-c2ccc(F)cc2)[nH]1] | 500 | 0 | 30 | 90 | 90 |
| 41 | 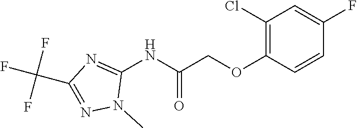 [SMILES: Cn1nc(C(F)(F)F)nc1NC(=O)COc1ccc(F)cc1Cl] | 500 | 40 | 60 | 70 | 95 |
| 40 | 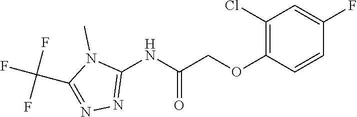 [SMILES: Cn1c(NC(=O)COc2ccc(F)cc2Cl)nnc1C(F)(F)F] | 500 | 60 | 70 | 100 | 100 |
| 39 | 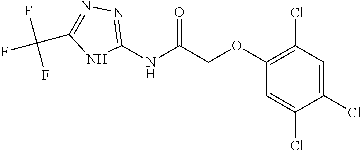 [SMILES: O=C(COc1cc(Cl)c(Cl)cc1Cl)Nc1nnc(C(F)(F)F)[nH]1] | 500 | 0 | 0 | 50 | 80 |
| 38 | 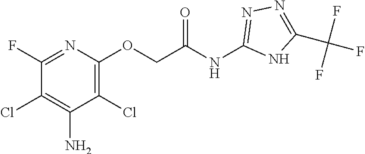 [SMILES: Nc1c(Cl)c(F)nc(OCC(=O)Nc2nnc(C(F)(F)F)[nH]2)c1Cl] | 500 | 30 | 0 | 98 | 100 |
| 37 | 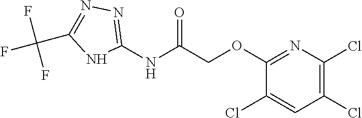 [SMILES: O=C(COc1nc(Cl)c(Cl)cc1Cl)Nc1nnc(C(F)(F)F)[nH]1] | 500 | 5 | 60 | 30 | 80 |
| 97 | 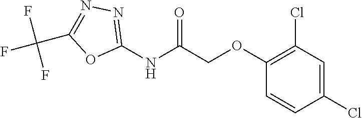 [SMILES: O=C(COc1ccc(Cl)cc1Cl)Nc1nnc(C(F)(F)F)o1] | 125 | 0 | 0 | 100 | 100 |
| 98 | 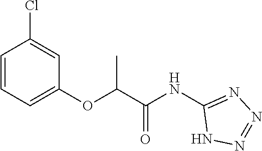 [SMILES: CC(Oc1cccc(Cl)c1)C(=O)Nc1nnn[nH]1] | 250 | 10 | 0 | 20 | 0 |
| 99 | 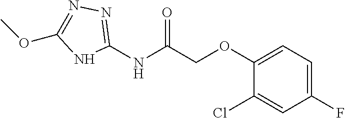 [SMILES: COc1nnc(NC(=O)COc2ccc(F)cc2Cl)[nH]1] | 250 | 0 | 0 | 50 | 50 |
| 100 | 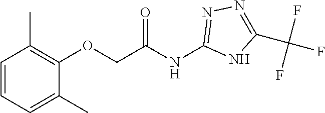 [SMILES: Cc1cccc(C)c1OCC(=O)Nc1nnc(C(F)(F)F)[nH]1] | 500 | 0 | 0 | 20 | 0 |
| 101 | 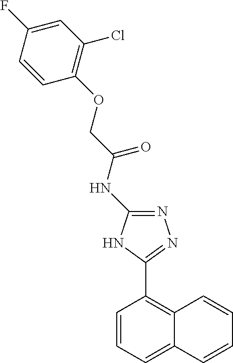 [SMILES: O=C(COc1ccc(F)cc1Cl)Nc1nnc(-c2cccc3ccccc23)[nH]1] | 500 | 20 | 70 | 80 | 100 |
| 102 | 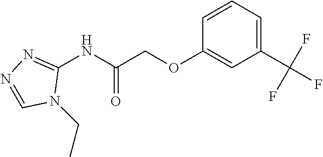 [SMILES: CCn1cnnc1NC(=O)COc1cccc(C(F)(F)F)c1] | 500 | 0 | 70 | 7 | 50 |

Pre-emergence testing was also performed using the same test species grown in a sandy loam soil mixture, Lolium multiflorum (LOLMU), Amaranthus retroflexus (AMARE), Echinochloa crus-galli (ECHCG), Veronica persica (VERPE).

After sowing, at growth stage BBCH 00-07 (pre-emergence), the plants are sprayed with an aqueous spray solution derived from the formulation of the technical active ingredient in acetone/water (50:50) solution containing 0.5% Tween 20 (polyoxyethlyene sorbitan monolaureate, GAS RN 9005-64-5). The test compounds are applied at the required 1a concentration of active ingredient in g/ha. The test plants are then grown in a glasshouse under controlled environmental conditions and watered regularly as required. After 14±1 days post-emergence in untreated plots (for the pre-emergence test), the test is evaluated by assessing the percentage damage caused to the plants in comparison with the untreated plots. The biological activities for pre-emergence testing are shown below (Table 4) in the following table as a % visual injury.

TABLE 4
Visual injury caused to plants from pre-emergence testing from a range of
compounds (NT = Not Tested).

| | | | LOLMU | ECHCG | VERPE | AMARE |
|---|---|---|---|---|---|---|
| | | | % | % | % | % |
| Compound | | Rate | visual | visual | visual | visual |
| number | Structure | g/ha | Injury | Injury | Injury | Injury |
| 95 | 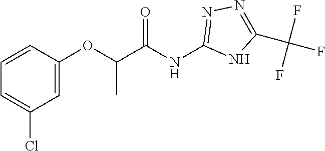 [SMILES: CC(Oc1cccc(Cl)c1)C(=O)Nc1nnc(C(F)(F)F)[nH]1] | 1000 | 0 | 0 | 40 | 0 |
| 94 | 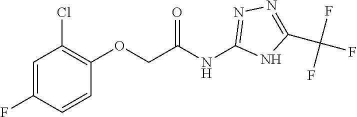 [SMILES: O=C(COc1ccc(F)cc1Cl)Nc1nnc(C(F)(F)F)[nH]1] | 1000 | 40 | 10 | 0 | 0 |
| 36 | 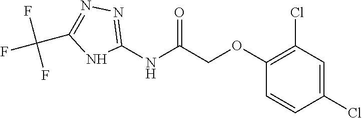 [SMILES: O=C(COc1ccc(Cl)cc1Cl)Nc1nnc(C(F)(F)F)[nH]1] | 1000 | 0 | 40 | 0 | 40 |
| 35 | 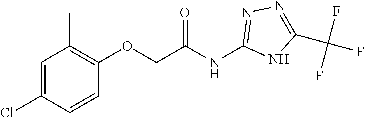 [SMILES: Cc1cc(Cl)ccc1OCC(=O)Nc1nnc(C(F)(F)F)[nH]1] | 1000 | 0 | 80 | 0 | 20 |
| 32 | 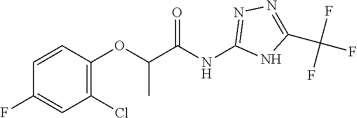 [SMILES: CC(Oc1ccc(F)cc1Cl)C(=O)Nc1nnc(C(F)(F)F)[nH]1] | 1000 | 0 | 0 | 5 | NT |
| 30 | 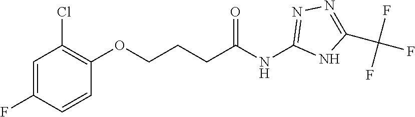 [SMILES: O=C(CCCOc1ccc(F)cc1Cl)Nc1nnc(C(F)(F)F)[nH]1] | 1000 | 60 | 0 | 0 | NT |
| 29 | 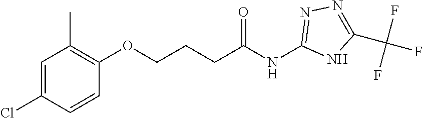 [SMILES: Cc1cc(Cl)ccc1OCCCC(=O)Nc1nnc(C(F)(F)F)[nH]1] | 1000 | 20 | 0 | 0 | NT |
| 28 | 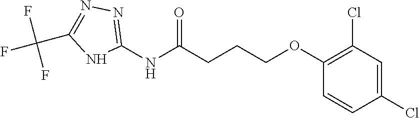 [SMILES: O=C(CCCOc1ccc(Cl)cc1Cl)Nc1nnc(C(F)(F)F)[nH]1] | 1000 | 40 | 0 | 0 | NT |
| 27 | 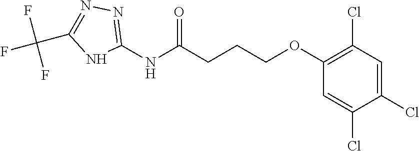 [SMILES: O=C(CCCOc1cc(Cl)c(Cl)cc1Cl)Nc1nnc(C(F)(F)F)[nH]1] | 1000 | 0 | 30 | 0 | NT |
| 26 | 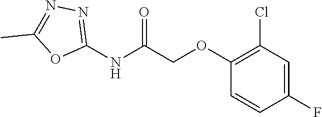 [SMILES: Cc1nnc(NC(=O)COc2ccc(F)cc2Cl)o1] | 1000 | 50 | 30 | 0 | NT |
| 25 | 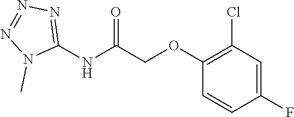 [SMILES: Cn1nnnc1NC(=O)COc1ccc(F)cc1Cl] | 1000 | 0 | 10 | 10 | NT |

Selected compounds of the invention were also assessed in comparative tests (Table 5) against structural analogues not according to the invention (e.g. a compound containing an imidazole and one containing an oxazole, neither of which fall within the scope of the claimed invention). In each test, compounds were applied at a rate of 125 g/ha. Test 1 compared the efficacy of a compound of the invention (93) with a non-claimed structure which contains an unsubstituted triazole (105). It is clear that compound 93 exhibits superior herbicidal activity. Test 2 compared the efficacy of compound 93 with compound 104, which contains an imidazole and is therefore not according to the invention. It is clear that compound 93 exhibits superior herbicidal activity. Test 3 compared the efficacy of compound 43, a further compound according to the invention, with compound 103, which contains an oxazole not according to the invention. It is clear that compound 43 exhibits superior herbicidal activity.

From the results of the tests in Table 5, it is clear that the particular 3-heteroatom heterocycles of the invention represent a significant improvement over the non-claimed 2-heteroatom heterocycles tested, and that an improvement is found in substituted triazoles compared to unsubstituted triazoles.

TABLE 5
Comparative tests of compounds of the invention against non-claimed structures with
structural differences

| | | | | LOLMU | ECHCG | VERPE | AMARE |
|---|---|---|---|---|---|---|---|
| | | | | % | % | % | % |
| Test | Compound | | Rate | visual | visual | visual | visual |
| No. | number | Structure | g/ha | Injury | Injury | Injury | Injury |
| 1 | 93 | 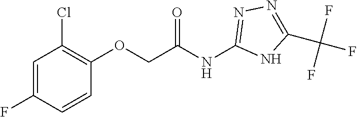 [SMILES: O=C(COc1ccc(F)cc1Cl)Nc1nnc(C(F)(F)F)[nH]1] | 125 | 20 | 30 | 80 | 60 |
| | 105 | 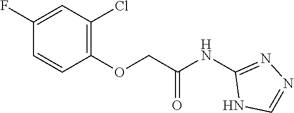 [SMILES: O=C(COc1ccc(F)cc1Cl)Nc1nnc[nH]1] | 125 | 0 | 20 | 40 | 50 |
| 2 | 93 | 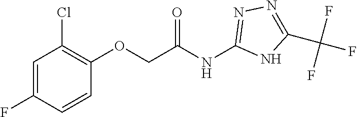 [SMILES: O=C(COc1ccc(F)cc1Cl)Nc1nnc(C(F)(F)F)[nH]1] | 125 | 20 | 30 | 80 | 60 |
| | 104 | 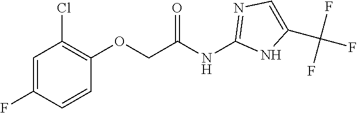 [SMILES: O=C(COc1ccc(F)cc1Cl)Nc1ncc(C(F)(F)F)[nH]1] | 125 | 10 | 30 | 70 | 40 |
| 3 | 43 | 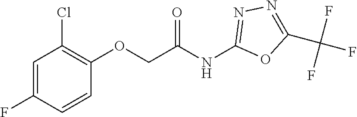 [SMILES: O=C(COc1ccc(F)cc1Cl)Nc1nnc(C(F)(F)F)o1] | 125 | 50 | 30 | 70 | 70 |
| | 103 | 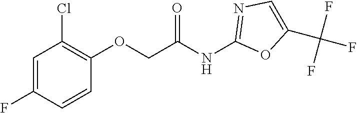 [SMILES: O=C(COc1ccc(F)cc1Cl)Nc1ncc(C(F)(F)F)o1] | 125 | 20 | 30 | 50 | 50 |

1. Use of a compound of general Formula (I) or an agriculturally acceptable salt thereof as an agrochemical: 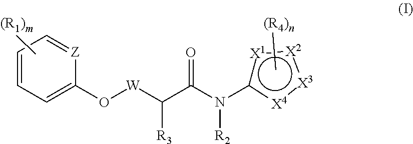
wherein
each R₁ is independently selected from CN, nitro, halogen, OR₆, SR₆, NR₆R₇, NR₆OR₇, NR₆NR₇R₈, ONR₆R₇, ON(═CR₆), R₂₀, OR₂₀, SR₂₀, NR₆R₂₀, C₁₋₆ alkyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, C₂-C₆ alkenyl and C₂₋₆ alkynyl, any of which may be optionally substituted;
wherein R₁ may, together with a further R₁ group, form a C₃-C₁₀ cycloalkyl, C₃₋₁₀ heterocycloalkyl, C₃₋₁₀ cycloalkenyl, C₃₋₁₀ heterocycloalkenyl, C₆-C₁₀ aryl or C₅-C₁₀ heteroaryl which may be optionally substituted;
R₂ is selected from H or C₁-C₄ alkyl;
R₃ is selected from H and optionally substituted C₁-C₄ alkyl,
W is selected from —(C(R₃)₂)ₚ— wherein p is 0, 1, 2 or 3;
Z is selected from CH, CR₁ and N;
X¹-X⁴ are independently selected from CH, CR₄, N, NH, NR₄ and O;
wherein at least three of X¹-X⁴ are N, NH, NR₄ or O;
such that the ring containing X¹-X⁴ is aromatic;
each R₄ is independently selected from the same groups as listed for R₁,
further wherein R₄ may together with a further R₄ group form a C₃-C₁₀ cycloalkyl, C₃₋₁₀ heterocycloalkyl, C₃₋₁₀ cycloalkenyl, C₃₋₁₀ heterocycloalkenyl, C₆-C₁₀ aryl or C₅-C₁₀ heteroaryl which may be optionally substituted;
R₆, R₇ and R₈ are independently selected from the group consisting of H, C₁₋₄ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl which may be optionally substituted; wherein R₆ may independently or together with R₇ form a C₃-C₁₀ cycloalkyl, C₃₋₁₀ heterocycloalkyl, C₃₋₁₀ cycloalkenyl, C₃₋₁₀ heterocycloalkenyl, C₆-C₁₀ aryl or C₅-C₁₀ heteroaryl which may be optionally substituted;
R₂₀ is selected from
C(═O)R₆,
C(═O)OR₆, C(═O)NR₆R₇, C(═O)NR₆C(═O)R₇, C(═O)C(═O)R₆, C(═O)C(═O)OR₆,
C(═O)C(═O)NR₆R₇, C(═O)NR₇S(═O)OR₆, C(═O)NR₆OR₇, (C═O)SR₆,
S(═O)R₆, S(═O)₂R₆, S(═O)OR₆, S(═O)₂OR₆, S(═O)NR₆R₇, S(═O)₂NR₆R₇,
S(═O)₂NR₇COR₆, S(═O)(═NRs)NR₆R₇, S(═O)(═NR₆)R₇, S(═NR₆)R₇,
SC(═O)R₆, SC(═O)OR₆, SC(═O)NR₆R₇,
C(═S)R₆, C(═S)OR₆, C(═S)NR₆R₇,
CR₇(═NR₆), CR₇(═N—OR₆), COR₇(═N—OR₆), CNR₇R₈(═N—OR₆), CR₈(═N—NR₇R₆);
m is an integer selected from 0, 1, 2, 3 or 4;
n is an integer selected from 1 or 2.

2. Use of a compound according to claim 1, wherein the optional substituents are selected from one or more of CN, nitro, halogen, OR₆, SR₆, NR₆R₇, NR₆OR₇, NR₆NR₇R₈, R₂₀, OR₂₀, SR₂₀, NR₆R₂₀, C₁₋₆ alkyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, C₂-C₆ alkenyl and C₂₋₆ alkynyl which may themselves be optionally substituted.

3. Use of a compound according to claim 1 wherein each R₄ is independently selected halogen, NH₂, NR₆R₇, SR₆, C₁₋₆ alkyl, C₁₋₆ haloalkyl, C₃-C₁₀ cycloalkyl, C₃-C₁₀-heterocycloalkyl, C₆₋₂₀ aryl, C₅₋₂₀ heteroaryl, C₁₋₆ alkoxy and C₁₋₆ carboxy, any of which may be optionally substituted; preferably wherein R₆ and R₇ are selected from H and C₁₋₄ alkyl which may themselves be optionally substituted with halo or hydroxyl.

4. Use of a compound according to claim 1 wherein each R₁ is independently selected from halogen, C₁₋₆ alkyl, C₁₋₆ haloalkyl, C₃₋₁₀ cycloalkyl and C₃₋₁₀ heterocycloalkyl which may themselves be optionally substituted.

5. Use of a compound according to claim 1 wherein three of X¹-X⁴ are N, NH, NR₄ or O, such that the ring containing X¹-X⁴ is aromatic.

6. Use of a compound according to claim 5 wherein three of X¹-X⁴ are selected from N, NH and NR₄, and the remaining X group is selected from CH and CR₄, such that the 5-membered ring including X¹-X⁴ is a triazole, wherein it is understood that when one of the three X¹-X⁴ selected from N, NH and NR₄ is NH, the remaining X group is CR₄.

7. Use of a compound according to claim 6, wherein the 5-membered ring including X¹-X⁴ is selected from:

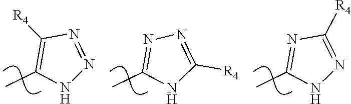

8. Use of a compound according to claim 5 wherein two of X¹-X⁴ are N and one of X¹-X⁴ is O, and the remaining X group is CR₄ such that the 5-membered ring including X¹-X⁴ is an oxadiazole.

9. Use of a compound according to claim 8 wherein the 5-membered ring including X¹-X⁴ is selected from:

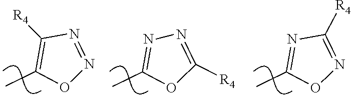

10. Use of a compound according to claim 1 wherein the compound is of general Formula (II): 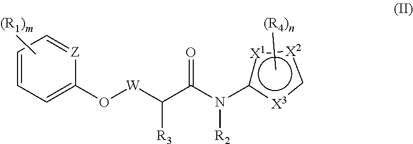
wherein
each R₁, R₂, R₃, W, Z, R₄, are as defined in claim 1;
X¹-X³ are independently selected from N, NH and NR₄;
wherein two of X¹-X³ are N;
such that the ring containing X¹-X³ is aromatic;
m is an integer selected from 0, 1, 2 or 3; and
n is an integer selected from 1 or 2.

11. Use of a compound according to claim 10 wherein the compound is of general Formula (III) 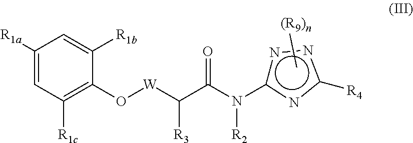
wherein
R₁ₐ, R_{1b} and R_{1c} are independently selected from hydrogen, halide, CN, NO₂, SO₂R₆, SR₆, C₁₋₆ alkyl, C₁₋₆ haloalkyl, C₁₋₆ alkoxy, C₁₋₆ carboxy, NR₆R₇;
R₂, R₃, W, R₄ are as defined in claim 10;
R₆ and R₇ are selected from H and C₁₋₄ alkyl which may themselves be optionally substituted with halo or hydroxyl;
R₉ is selected from hydrogen, C₁₋₄ alkyl or C₃₋₆ cycloalkyl;
n is equal to 1.

12. Use of a compound according to claim 11 wherein R₄ is selected from C₁₋₆ alkyl, C₁₋₆ haloalkyl and C₃₋₈ cycloalkyl, preferably wherein the halide in C₁₋₆ haloalkyl is fluoride.

13. Use of a compound according to claim 11 wherein R₁ₐ and R_{1b} are selected from methyl and halide, preferably from halide.

14. Use of a compound according to claim 11 wherein R_{1c} is hydrogen.

15. Use of a compound according to claim 11 wherein R₁ₐ is chloride or fluoride and R_{1b} is chloride.

16. Use of a compound according to claim 10 wherein the compound is selected from the following formulae:

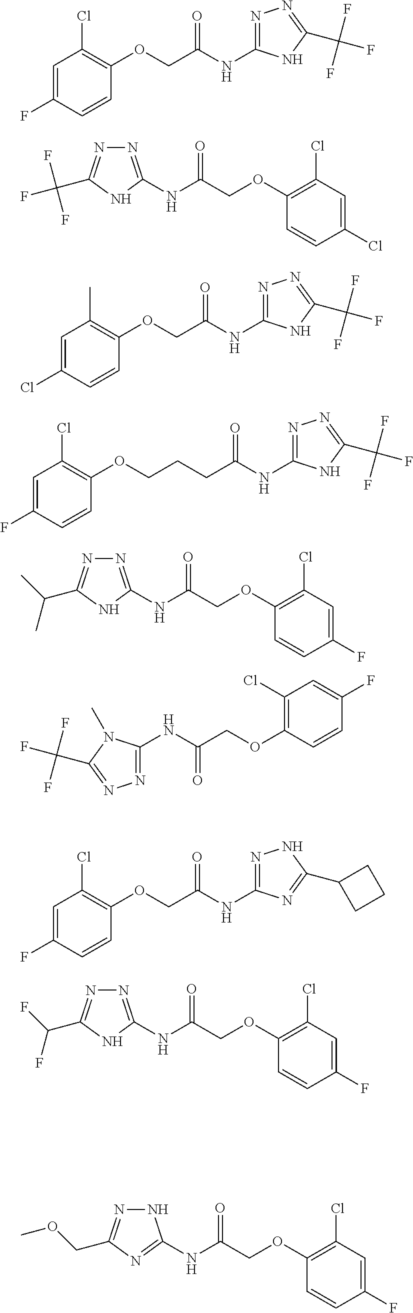

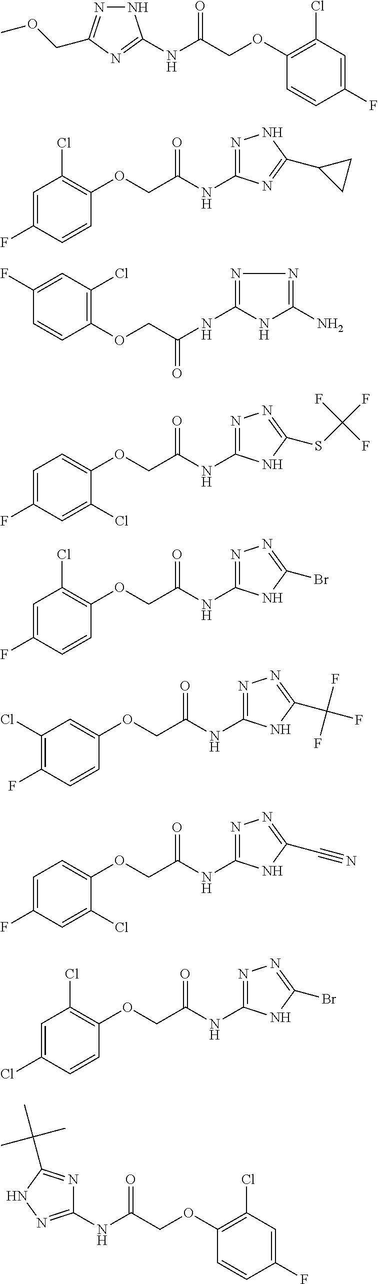

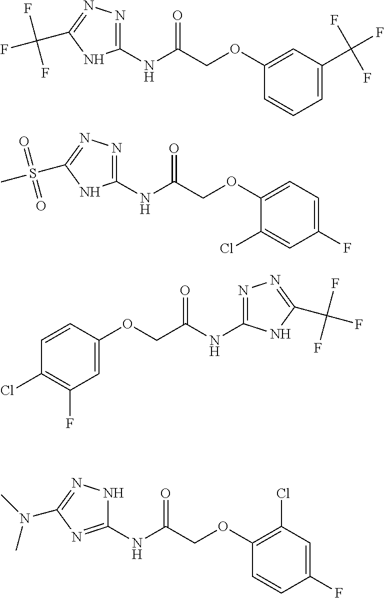

17. Use of a compound according to claim 1 wherein the compound is of general Formula (VI): 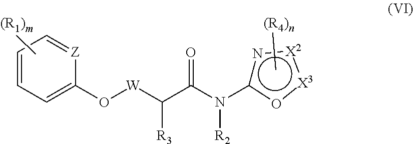
wherein
each R₁, R₂, R₃, W, Z, R₄ are as defined in claim 1;
X²-X³ are independently selected from N and CR₄;
wherein one of X²-X³ is N;
m is an integer selected from 0, 1, 2 or 3; and
n is an integer selected from 1 or 2.

18. Use of a compound according to claim 17 wherein the compound is of general Formula (VII): 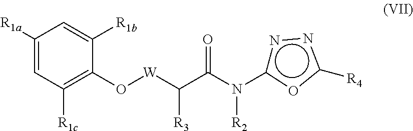
wherein
R₁ₐ, R_{1b} and R_{1c} are independently selected from hydrogen, halide, CN, NO₂, SO₂R₆, SR₆, C₁₋₆ alkyl, C₁₋₆ haloalkyl, C₁₋₆ alkoxy, C₁₋₆ carboxy, NR₆R₇;
R₂, R₃, R₄, W are as defined in claim 17;
R₆, R₇ are as defined in claim 1.

19. Use of a compound according to claim 17 wherein the compound is of general Formula (VIII): 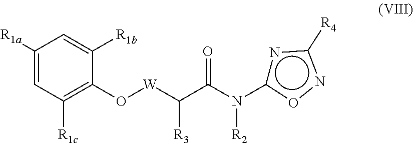
wherein
R₁ₐ, R_{1b} and R_{1c} are independently selected from hydrogen, halide, CN, NO₂, SO₂R₆, SR₆, C₁₋₆ alkyl, C₁₋₆ haloalkyl, C₁₋₆ alkoxy, C₁₋₆ carboxy, NR₆R₇;
R₂, R₃, R₄, W are as defined in claim 17;
R₆, R₇ are as defined in claim 1.

20. Use of a compound according to claim 17 wherein R₄ is selected from C₁₋₆ alkyl, C₁₋₆ haloalkyl and C₃₋₈ cycloalkyl, preferably wherein the halide in C₁₋₆ haloalkyl is fluoride.

21. Use of a compound according to claim 18 wherein R₁ₐ and R_{1b} are selected from methyl and halide, preferably halide.

22. Use of a compound according to claim 18 wherein R_{1c} is hydrogen.

23. Use of a compound according to claim 18 wherein R₁ₐ is chloride or fluoride and R_{1b} is chloride.

24. Use of a compound according to claim 17 wherein the compound is selected from the following formulae:

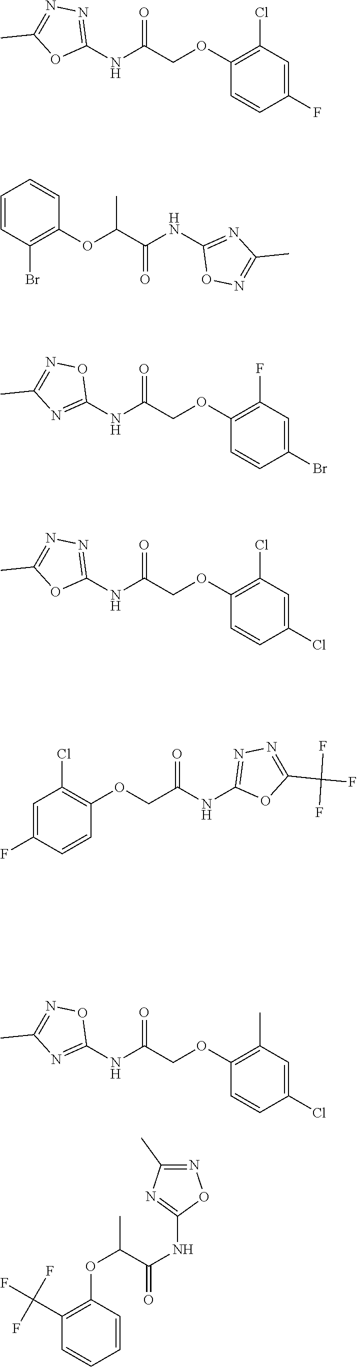

25. Use of a compound according to claim 1 as a herbicide.

26. An agrochemical composition comprising a compound as defined in claim 1 and an agriculturally acceptable formulation adjuvant.

27. An agrochemical composition according to claim 26 further comprising at least one additional pesticide.

28. An agrochemical composition according to claim 27 wherein the at least one additional pesticide is a herbicide or herbicide safener.

29. An agrochemical composition according to claim 26 which is a herbicidal composition.

30. A method of controlling weeds at a locus comprising application to the locus of a weed controlling amount of a composition according to claim 26.

31. A compound selected from the following formulae: 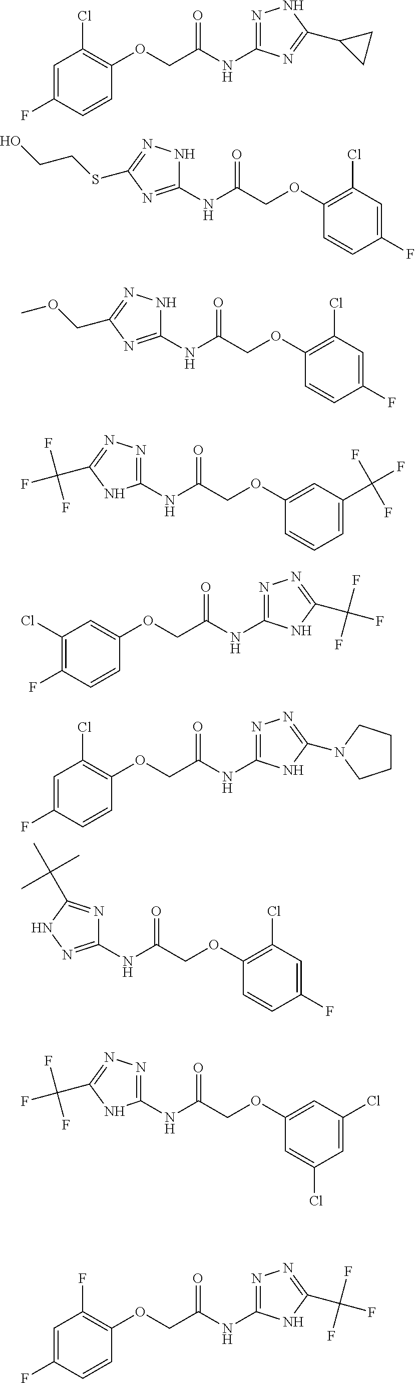 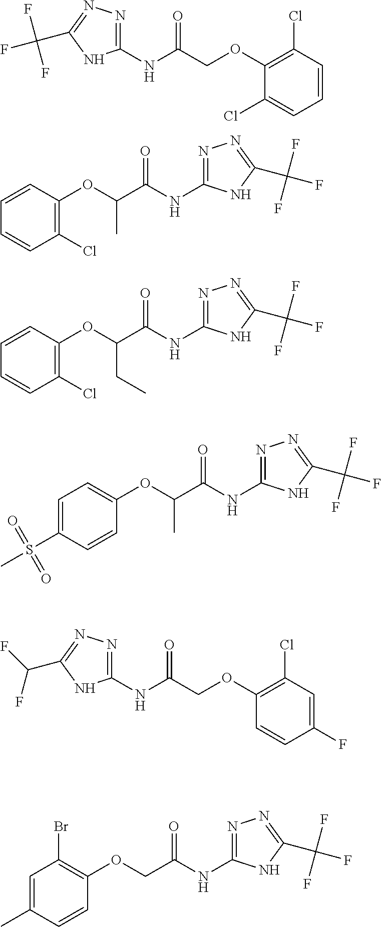 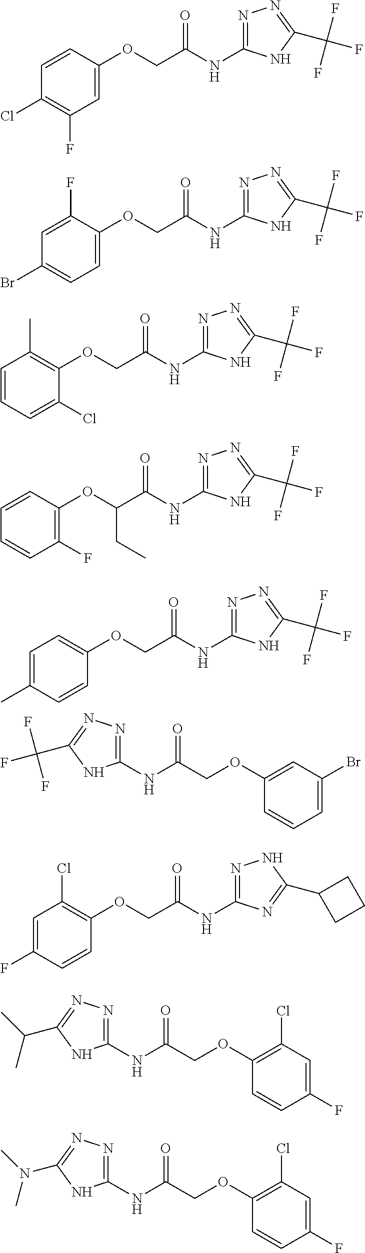 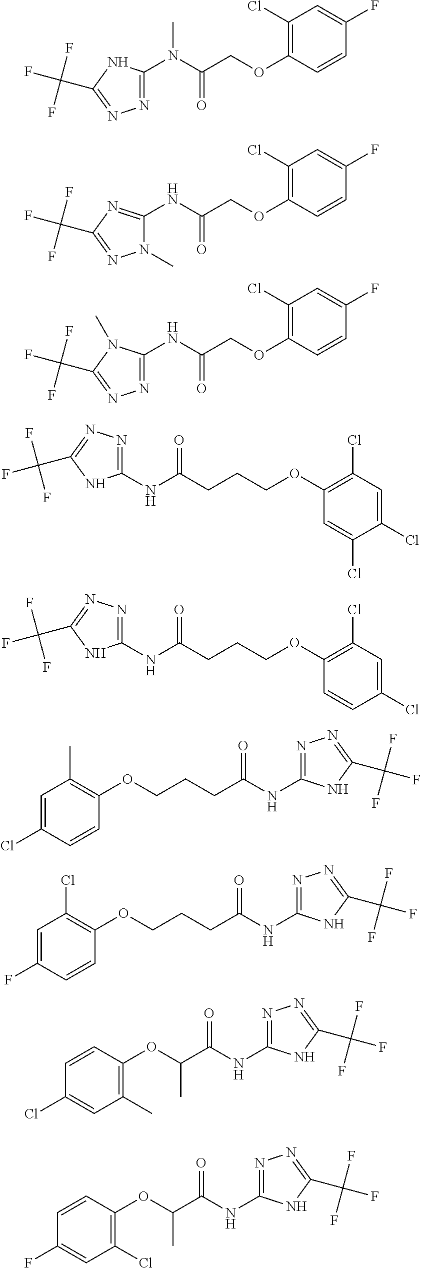 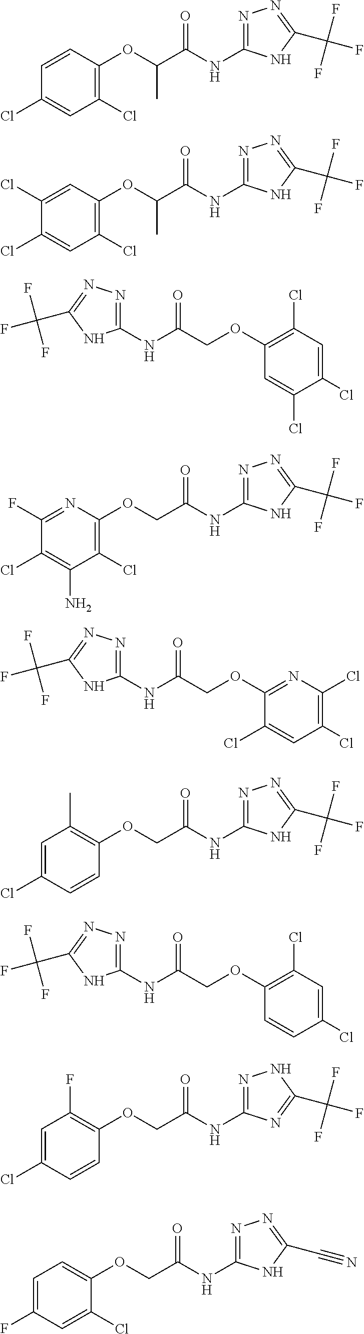 [chemical structure]
